(19) United States
(12) Patent Application Publication
(10) Pub. No.: US 2008/0125435 A1
(43) Pub. Date: May 29, 2008

(54) 1-(Het)aryl-3-[hetaryl-piperidin-4-yl]-thioureas as modulators of the EP2 receptor

(76) Inventors: Nico Brauer, Jena (DE); Bernd Buchmann, Hohen Neuendorf (DE); Christoph Huwe, Berlin (DE); Marcus Koppitz, Berlin (DE); Antonius Ter Laak, Berlin (DE); Gernot Langer, Falkensee (DE); Bernhard Lindenthal, Berlin (DE); Olaf Peters, Tabarz (DE); Tim Wintermantel, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/896,924

(22) Filed: Sep. 6, 2007

Related U.S. Application Data
(60) Provisional application No. 60/842,680, filed on Sep. 7, 2006.

(30) Foreign Application Priority Data
Sep. 7, 2006 (EP) 06090158.4

(51) Int. Cl. A61K 31/502; C07D 401/04; A61K 31/517; A61P 15/00; A61P 19/10; A61P 35/04; A61P 31/00; A61K 31/473

(52) U.S. Cl. 514/252.03; 546/194; 514/313; 514/266.22; 544/237; 544/284

(57) ABSTRACT

The present invention relates to aryl-3-[(het)aryl-piperidin-4-yl]-thioureas, processes for the production thereof and the use thereof for the production of pharmaceutical agents for the treatment of diseases and indications which are connected with the EP₂ receptor.

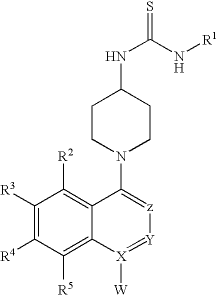

This application claims the benefit of the filing date of U.S. Provisional Application Ser. No. 60/842,680 filed Sep. 7, 2006, which is incorporated by reference herein.

The present invention relates to 1-(het)aryl-3-[hetaryl-piperidin-4-yl]-thioureas as EP₂ receptor modulators, processes for the production thereof and the use thereof as medicaments.

It has already long been known that prostaglandins are the key molecules in the processes of female reproductive biology, such as for example the regulation of ovulation, fertilization, nidation, decidualization (e.g. placenta formation) and menstruation. Prostaglandins also play an important part in pathological changes in the reproductive tract, including hypermenorrhea, dysmenorrhea, endometriosis and cancer. So far, the mechanism through which the prostaglandins cause these changes has not yet been completely elucidated. Recent findings indicate that prostaglandins, their receptors and their signal transduction routes are involved in processes such as angiogenesis, apoptosis, proliferation and in inflammatory/antiinflammatory and immunological processes.

The effects of the prostaglandins are mediated by their G-protein-coupled receptors, which are located on the cell surface. Of particular interest is prostaglandin E₂ (PGE₂), which brings about a great variety of cellular actions since it binds to functionally different receptor subtypes, namely the EP₁, EP₂, EP₃ and EP₄ receptors. The receptor subtypes to which prostaglandin E₂ binds appear to be of particular interest for the receptor-mediated effects which are involved in fertility regulation. Thus it could be shown that the reproductive functions are affected in EP₂ knockout mice (EP₂^{-/-}), i.e. in mice which no longer carry the PGE₂ receptor subtype EP₂, and that these animals have a lower “litter number” (Matsumoto et al., 2001, Biology of Reproduction 64, 1557-1565). Likewise, it could be shown that these EP₂ knockout mice (Hizaki et al. Proc Natl Acad Sci U.S.A. 1999 Aug. 31; 96 (18), 10501-10506) show markedly reduced cumulus expansion and severe subfertility, which demonstrates the importance of the prostaglandin EP₂ receptor for this process. Accordingly, the EP₂ receptor is an important target for the development of drugs for the regulation of female fertility. The existence of the four subclasses of the PGE₂ receptor opens up the possibility of targeted development of selectively acting compounds. However, hardly any selective EP₂ receptor ligands which bind to the EP₂ subtypes of the PGE₂ receptor were previously known, since most known compounds also bind to the other PGE₂ receptor subtypes, for example to the EP₄ receptor.

EP₂ receptor antagonists are for example described in the application US2005059742 (Jabbour, Medical Research Council). A method is claimed wherein an EP₂- and/or an EP₄-antagonist can be used for the treatment of hypermenorrhea and dysmenorrhea. AH6809 is disclosed as an antagonist of the EP₂- or EP₄ receptor, and no other specific antagonists and no new compounds are disclosed.

In a previous application, EP₂ or EP₄ antagonists for the treatment of pathological states, such as for example uterine carcinoma, myoma and endometriosis, are claimed by the same group (EP 1467738). Similarly, no new compounds are disclosed.

In the application WO03/016254, Ono Pharmaceutical claims the production of benzoic acid or saturated carboxylic acid derivatives which are substituted with aryl or heterocycles, inter alia as PGE₂ receptor antagonists. The compounds disclosed are claimed for the treatment of a large number of diseases, among them also allergic diseases, Alzheimers disease, pain, abortion, menstrual problems, hypermenorrhea and dysmenorrhea, endometriosis, diseases of the bones, ischemia and the like. The compounds described are however characterized by particularly high affinity to the EP₃ receptor. In a further application (WO04/032964), new compounds are described which likewise are characterized by particularly high affinity to the EP₃ receptor, which also find use as EP₂ antagonists for the treatment and prophylaxis of allergic diseases.

In the application WO04/39807 by the company Firma Merck Frosst, Canada, the production of pyridopyrrolizines and pyridoindolizines is disclosed. These compounds are however characterized by good binding to the PGD₂ receptor; this receptor represents another subtype of the prostaglandin receptor.

Naphthalene derivates as EP₄ receptor ligands are disclosed by the SmithKline Beecham Corporation in the application US2004102508. The compounds claimed find their use in the treatment or prophylaxis of pain, allergic reactions and neurodegenerative diseases.

EP₄ antagonists (γ-lactams) are claimed in the application WO03/103604 (Applied Research Systems). The compounds bind ca. 60 times better to the EP₄ than to the EP₂ receptor and are inter alia claimed for the treatment of premature labor, dysmenorrhea, asthma, sterility or fertility disorders. In the applications WO03/053923 (substituted pyrrolidines) or WO03/035064 (substituted pyrazolidiones), the same company claims compounds for the treatment of diseases, which are associated with prostaglandins, such as for example infertility, hypertension and osteoporosis. The compounds bind to the EP₄- and EP₂ receptor subtypes. In the application WO03/037433, ω-cycloalkyl, 17-heteroaryl-prostaglandin derivatives are claimed as EP₂ receptor antagonists, in particular for the treatment of elevated intraocular pressure.

In the application WO03/064391 (Pfizer Products), metabolites of [3-[[N-(4-tert-butylbenzyl)(pyridin-3-ylsulfonyl)amino]methyl]acetic acid are described which inhibit the binding of [³H]-prostaglandin E₂ to the EP₂ receptor. The use of these metabolites for the treatment of osteoporosis is disclosed.

In the application US2005124577, Tani et al. claim 8-azaprostaglandin derivatives for the treatment of immunological diseases, allergic diseases, premature labor, abortion and the like. The compounds bind to the EP₂ and EP₄ receptor.

In the European patent EP 1306087, EP₂ receptor agonists are described which find their use in the treatment of erectile dysfunction. The same structural class is described in the European patent EP 860430 and the use thereof for the production of a drug for the treatment of immunological diseases, asthma and abortion is claimed. The application WO04/32965 describes the EP₂ receptor agonists which are used for the treatment and prevention of diseases which are caused by organ failure due to ischemia. In WO04/009117, EP₂ and EP₄ receptor agonists are described for the treatment of diseases which are caused by uterine contraction, for example menstrual problems.

In the applications WO 03/74483 and WO03/09872, agonists are described which bind equally to the EP₂ and the EP₄ receptor (Ono Pharmaceuticals).

The agonists of the EP₂ and EP₄ receptors are often described in connection with the treatment of osteoporosis (WO99/19300, US2003/0166631, WO03/77910, WO03/45371, WO 03/74483 and WO03/09872) and for the treatment of glaucoma (WO04/37813, WO04/37786, WO04/19938, WO03/103772, WO03/103664, U.S. Pat. No. 6,747,037, U.S. Pat. No. 6,410,591, WO03/40123, WO03/47513, WO03/47417).

In the patent application WO04/12656, EP₂ receptor agonists are claimed in connection with inflammation.

In the patent application WO03/77919, EP₄ receptor agonists are claimed for fertility treatment.

Hitherto, however, no selective EP₂ receptor agonists and antagonists which regulate the processes which are ultimately responsible for nidation and decidualization, and thus contribute to the promotion or inhibition of fertility were known.

This leads to the objective of providing stable and effective compounds which bind selectively to the EP₂ receptor for the development of new drugs.

Surprisingly, it has now been found that compounds of the general formula I

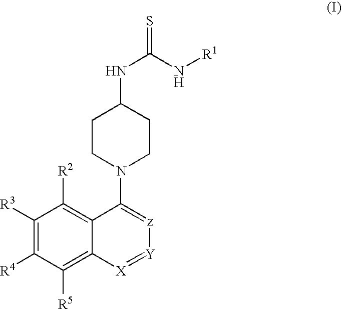

wherein
X, Y, Z independently of each other mean a nitrogen residue or a carbon residue —C—R⁸, wherein R⁸ can be hydrogen or a C₁-C₄ alkyl residue,
   under the condition that at least one, but at most 2 of the groups X, Y and Z is a nitrogen residue,
R¹ means a 5-12-membered, mono- or bicyclic aryl or heteroaryl ring, which can be unsubstituted or optionally singly to triply substituted,
R²-R⁵ independently of each other mean a hydrogen, halogen, cyano,
   or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which can be unsubstituted or optionally substituted
   a C₃-C₁₀ cycloalkyl ring, which can be unsubstituted or optionally substituted,
   a C₂-C₆ alkenyl or C₂-C₆ alkynyl group, which can be unsubstituted or optionally substituted,
   a 5-12-membered, mono- or bicyclic aryl or heteroaryl ring, which can be unsubstituted or optionally substituted,
R⁶, R⁷ mean a hydrogen, a C₁-C₆ alkyl, a C₃-C₁₀ cycloalkyl ring, a 5-12-membered mono- or bicyclic aryl or heteroaryl ring, wherein the alkyl, cycloalkyl and (het)aryl groups can be unsubstituted or optionally substituted or
R⁶, R⁷ together form a 3-8-membered ring,
   and isomers and salts thereof and cyclodextrin clathrates thereof, can overcome the known disadvantages and achieve better selectivity to the EP₂ receptor and thus better efficacy and longer duration of action.

The saturated, unbranched C₁-C₄ alkyl substituents stated under R⁸ to R¹⁰ are for example a methyl, ethyl, n-propyl or n-butyl, and the branched C₃-C₄ alkyl groups an iso-propyl, iso-butyl, sec-butyl or tert-butyl group.

The alkyl groups can optionally be singly to multiply substituted with halogen atoms, e.g. fluorine, chlorine or bromine.

The saturated, unbranched C₁-C₆ alkyl substituents stated under R² to R⁷ are for example a methyl, ethyl, n-propyl, n-butyl, n-pentyl or n-hexyl, and the branched C₃-C₆ alkyl groups an iso-propyl, iso-butyl, sec-butyl, tert-butyl, iso-pentyl, neo-pentyl, 2-methylpentyl, 2,2-dimethylbutyl- or 2,3-dimethylbutyl group.

The alkyl groups can optionally be singly to quintuply substituted with halogen atoms (e.g. fluorine, chlorine or bromine), and with cyano, hydroxy, amino or carboxyl groups.

The C₂-C₆ alkenyl substituents in R²-R⁵ and/or the C₂-C₄ alkenyl substituents in R⁹-R¹⁰ are each straight-chain or branched, wherein for example the following residues are meant:

Vinyl, allyl, homoallyl, (E)-but-2-enyl, (Z)-but-2-enyl, pent-4-enyl, (E)-pent-3-enyl, (Z)-pent-3-enyl, (E)-pent-2-enyl, (Z)-pent-2-enyl, 2-methylvinyl, 3-methylbut-3-enyl, 2-methylbut-3-enyl, (E)-2-methylbut-2-enyl, (Z)-2-methylbut-2-enyl, 2-ethylprop-2-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, 1-methylpent-4-enyl, (E)-1-methylpent-3-enyl, (Z)-1-methylpent-3-enyl, 1-ethylbut-3-enyl, (E)-1-methylpent-2-enyl and (Z)-1-methylpent-2-enyl.

The alkenyl groups can optionally be singly to triply substituted with halogen atoms (e.g. fluorine, chlorine or bromine), and with cyano or carboxyl groups.

The C₂-C₆ alkynyl substituents in R²-R⁵ and/or the C₂-C₄ alkenyl substituents in R⁹-R¹⁰ are each straight-chain or branched, wherein for example the following residues are meant: ethinyl, prop-1-in-1-yl, but-1-in-1-yl or but-2-in-1-yl.

The alkynyl groups can optionally be singly substituted with halogen atoms (e.g. fluorine, chlorine or bromine), and with cyano or carboxyl groups.

Halogen is understood to mean the following: fluorine, chlorine, bromine or iodine.

The C₃-C₁₀ cycloalkyl substituents stated in R²-R⁷ and the C₃-C₆ cycloalkyl substituents stated in R⁹-R¹⁰ should be understood to mean cycloalkyl rings such as cyclopropyl, cyclobutyl, cyclopentyl, cyclohexyl, cycloheptyl, cyclooctyl, adamantyl or decalinyl.

The cycloalkyl groups can optionally be singly to doubly substituted with halogen atoms (fluorine, chlorine or bromine), and with cyano, hydroxy, amino or carboxyl groups.

The 5-12-membered, mono- or bicyclic aryl or heteroaryl residue stated in R¹ to R⁷, which can optionally be singly or triply substituted, are understood to mean 5-12-membered ring systems, which instead of the carbon can contain one or more, similar or different hetero atoms, such as oxygen, nitrogen or sulfur in the ring, can be mono- or bicyclic and in addition can each be benzo-condensed and are connected to the structure via one of the possible linkage sites.

For example, as a 5-12-membered, mono- or bicyclic aryl residue, the following may be named: cyclopentadienyl, phenyl, tropyl, cyclooctadienyl, indenyl, naphthyl, azulenyl or biphenyl.

The 5-12-membered, mono- or bicyclic heteroaryl groups can be a pyridinyl, pyrimidinyl, quinolinyl, isoquinolinyl, phthalazinyl, quinazolinyl, quinoxalinyl, cinnolinyl, benzofuranyl, benzothiophenyl, 1,3-benzodioxolyl, benzimidazolyl, 2,1,3-benzothiadiazolyl, indolyl, furanyl, thiophenyl, oxazolyl, isoxazolyl, thiazolyl, pyrrolyl, pyrazolyl, pyrazinyl, pyridazinyl, triazolyl, tetrazolyl or imidazolyl group linked via one of the substitutable positions.

The 5-6-membered aryl or heteroaryl residue, which can optionally be singly or triply substituted, stated in R⁹ to R¹⁰ is understood to mean 5-6-membered ring systems, which instead of the carbon can contain one or more, similar or different hetero atoms, such as oxygen, nitrogen or sulfur in the ring and are bound to the structure via one of the possible linkage sites.

For example as a 5-6-membered, mono- or bicyclic aryl residue the following may be named: cyclopentadienyl or phenyl.

The 5-6-membered heteroaryl groups can be a pyridinyl, pyrimidinyl, furanyl, thiophenyl, oxazolyl, isoxazolyl, thiazolyl, pyrrolyl, pyrazolyl, pyrazinyl, pyridazinyl or imidazolyl group linked via one of the substitutable positions.

The 3-8-membered ring which can be formed by ring closure of R⁶ and R⁷ or R⁹ and R¹⁰ can be a cycloalkyl or a nitrogen-containing heterocycle. As examples of a 3-8-membered cycloalkyl ring the, following may for example be named: cyclopropyl, cyclobutyl, cyclopentyl, cyclohexyl, cycloheptyl, or cyclooctyl.

As examples of an 3-8-membered, nitrogen-containing heterocycle the following may for example be named: aziridinyl, azetidinyl, pyrrolidinyl, piperidinyl, piperazinyl, morpholinyl, azepanyl or [1,4]-diazepanyl.

The free alcohols of the compounds according to the invention can also be present as esters and thus are prodrugs of the physiological compounds of the general formula I, which in the body metabolize to compounds of the general formula I.

Suitable compounds are for example listed in Hans Bundgaard (Ed.), Design of Prodrugs, Elsevier, Amsterdam 1985.

If an acid function is present, the physiologically compatible salts of organic and inorganic bases are suitable as salts, such as for example the alkali metal and alkaline earth salts of good solubility and N-methyl-glucamine, dimethyl-glucamine, ethylglucamine, lysine, 1,6-hexadiamine, ethanolamine, glucosamine, sarcosine, serinol, tris-hydroxymethylaminomethane, aminopropanediol, Sovak base and 1-amino-2,3,4-butanetriol.

If a basic function is present, as inorganic acids inter alia hydrochloric acid, hydrobromic acid, sulfuric acid, phosphoric acid or nitric acid, as carboxylic acids inter alia acetic acid, propionic acid, hexanoic acid, octanoic acid, decanoic acid, oleic acid, stearic acid, maleic acid, fumaric acid, succinic acid, benzoic acid, ascorbic acid, oxalic acid, salicylic acid, tartaric acid, citric acid, lactic acid, glycolic acid, malic acid, mandelic acid, cinnamic acid, glutamic acid and aspartic acid, and as sulfonic acids inter alia methanesulfonic acid, ethanesulfonic acid, toluenesulfonic acid, benzenesulfonic acid and naphthalenesulfonic acid are possibilities for the formation of physiologically compatible salts of the compounds according to the invention of the general formula I, by the methods known to the person skilled in the art.

Preferred are the compounds of the general formula I, wherein
X means a nitrogen residue,
Y, Z mean a carbon residue —C—R⁸,
R⁸ means a hydrogen or a C₁-C₄ alkyl residue,
R¹ means a 5-12-membered, mono- or bicyclic, aryl or heteroaryl ring, which can be unsubstituted and optionally singly to triply substituted,
R²-R⁵ independently of each other mean a hydrogen, halogen, cyano,
   or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which is unsubstituted or can optionally be substituted,
   a C₃-C₁₀ cycloalkyl ring, which is unsubstituted or can optionally be substituted,
   a C₂-C₆ alkenyl- or C₂-C₆ alkynyl group, which is unsubstituted or can optionally be substituted,
   a 5-12-membered mono- or bicyclic aryl or heteroaryl ring, which is unsubstituted or can optionally be substituted,
R⁶, R⁷ means a hydrogen, a C₁-C₆ alkyl, a C₃-C₁₀ cycloalkyl ring or a 5-12-membered mono- or bicyclic aryl or heteroaryl ring, wherein the alkyl, cycloalkyl and (het)aryl groups are unsubstituted or can optionally be substituted or
R⁶, R⁷ together form a 3-8-membered ring.

Likewise preferred are the compounds of the general formula I, wherein
Y and Z mean a nitrogen residue,
X means a carbon residue —C—R⁸,
R⁸ means a hydrogen or a C₁-C₄ alkyl residue,
R¹ means a 5-12-membered mono or bicyclic aryl or heteroaryl ring, which is unsubstituted or can optionally be singly to triply substituted,
R²-R⁵ independently of each other mean hydrogen, halogen, cyano,
   or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which is unsubstituted or can optionally be substituted,
   a C₃-C₁₀ cycloalkyl ring, which is unsubstituted or can optionally be substituted,
   a C₂-C₆ alkenyl or C₂-C₆ alkynyl group, which is unsubstituted or can optionally be substituted,
   a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered mono- or bicyclic aryl or heteroaryl ring, wherein the alkyl, cycloalkyl and (het)aryl groups are unsubstituted or can optionally be substituted or
R⁶, R⁷ together form a 3-8-membered ring.

Likewise preferred are the compounds of the general formula I, wherein
X and Z each mean a nitrogen residue,
Y means a carbon residue —C—R⁸,
R⁸ means a hydrogen or a C₁-C₄ alkyl residue,
R¹ means a 5-12-membered mono- or bicyclic aryl or heteroaryl ring, which is unsubstituted and can optionally be singly to triply substituted,
R²-R⁵ independently of each other mean a hydrogen, halogen, cyano,
   or a OR⁶, OC(O)R⁶, S(O)ₙR⁶, wherein n=0, 1 or 2, SO₂NHR⁶, SO₂NHC(O)R⁶, NR⁶R⁶, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which is unsubstituted or can optionally be substituted,
   a C₃-C₁₀ cycloalkyl ring, which is unsubstituted or can optionally be substituted,
   a C₂-C₆ alkenyl or C₂-C₆ alkynyl group, which is unsubstituted or can optionally be substituted,
   a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered, mono- or bicyclic aryl or heteroaryl ring, wherein the alkyl, cycloalkyl and (het)aryl groups are unsubstituted or can optionally be substituted or
R⁶, R⁷ together form a 3-8-membered ring.

Likewise preferred are the compounds of the general formula I, wherein
X and Y each mean a carbon residue —C—R⁸,
Z means a nitrogen residue,
R⁸ means a hydrogen or a C₁-C₄ alkyl residue,
R¹ means a 5-12-membered or mono- or bicyclic aryl or heteroaryl ring, which is unsubstituted or can optionally be singly to triply substituted,
R²-R⁵ independently of each other mean a hydrogen, halogen, cyano,
   or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁷ or C(O)NR⁶R⁷ group,
   a C₁-C₆ alkyl group, which is unsubstituted or can optionally be substituted,
   a C₃-C₁₀ cycloalkyl ring, which is unsubstituted or can optionally be substituted,
   a C₂-C₆ alkenyl or C₂-C₆ alkynyl group, which is unsubstituted or can optionally be substituted,
   a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered, mono- or bicyclic aryl or heteroaryl ring, wherein the alkyl, cycloalkyl and (het)aryl groups are unsubstituted or can optionally be substituted or
R⁶, R⁷ together form a 3-8-membered ring.

Likewise preferred are the compounds of the general formula 1, wherein
X means a nitrogen residue,
Y, Z mean a carbon residue —C—R⁸,
R⁸ means a hydrogen,
R¹ means an unsubstituted 5-12-membered mono- or bicyclic aryl or heteroaryl ring, which is optionally singly to triply substituted,
   wherein the substituents can be selected from the group halogen,
   —C₁-C₄ alkyl, which is unsubstituted and can optionally be substituted,
   —OR⁶, —OC(O)R⁶, —S(O)ₙR⁶, wherein n=0, 1 or 2,
   —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
—R² means a hydrogen,
R³-R⁵ independently of each other mean a hydrogen, halogen, cyano, or a OR⁶, OC(O)R⁶, S(O)NR⁶ wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which is optionally unsubstituted or substituted,
   a C₃-C₁₀ cycloalkyl ring, which is optionally unsubstituted or substituted,
   a C₂-C₆ alkenyl or C₂-C₆ alkynyl group, which is optionally substituted or unsubstituted,
   a 5-12-membered mono- or bicyclic aryl or heteroaryl ring, which is optionally singly or multiply substituted,
      wherein the substituents can be selected from the group
         halogen,
         —C₁-C₄ alkyl, which is unsubstituted and can optionally be substituted,
         —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, a C₁-C₆ alkyl group, which is unsubstituted or can optionally be up to quintuply halogenated,
   a C₃-C₈ cycloalkyl residue,
   a 5-12-membered, mono or bicyclic aryl or heteroaryl ring, which is optionally singly or multiply substituted,
      wherein the substituents can be selected from the group
         halogen,
         cyano,
         R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or —C(O)—N—R⁹R¹⁰,
      wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
   a C₁-C₄ alkyl group, which is unsubstituted or can optionally be up to quintuply fluorinated,
   a C₂-C₄ alkenyl group, which is unsubstituted or can optionally be up to triply fluorinated,
   a C₂-C₄ alkynyl group, which is unsubstituted or can optionally be singly fluorinated,
   a C₃-C₆ cycloalkyl group,
   a 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which is unsubstituted or can be up to doubly substituted with fluorine, chlorine or trifluoromethyl, or
R⁹, R¹⁰ together form a 3-8-membered ring.

Likewise preferred are the compounds of the general formula I, wherein
Y and Z mean a nitrogen residue,
X means a carbon residue —C—R⁸,
R⁸ means a hydrogen or a methyl group,
R¹ a 5-12-membered mono- or bicyclic aryl or heteroaryl ring, which is optionally singly to triply substituted,
   wherein the substituents can be selected from the group halogen,
   —C₁-C₄ alkyl, which is unsubstituted and can optionally be substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano,
   or a OR⁶, OC(O)R⁶, S(O)NR⁶, wherein n=0, 1 or 2, SO₂NHR⁶, SO₂NHC(O)R⁶, NR⁶R⁶, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   a C₁-C₆ alkyl group, which is unsubstituted or can optionally be substituted,
   a C₃-C₁₀ cycloalkyl ring, which is unsubstituted or can optionally be substituted,
   a C₂-C₆ alkenyl or C₂-C₆ alkynyl group, which is unsubstituted or can optionally be substituted,
   an unsubstituted 5-12-membered mono- or bicyclic aryl or heteroaryl ring, which can optionally be singly or multiply substituted,
   wherein the substituents can be selected from the group halogen,
   —C₁-C₄ alkyl, which is unsubstituted and can optionally be substituted,
   —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
—R⁶, R⁷ mean a hydrogen, an unsubstituted C₁-C₆ alkyl group, which can optionally be up to quintuply halogenated,
   a C₃-C₈ cycloalkyl residue,
   an unsubstituted 5-12-membered, mono or bicyclic aryl or heteroaryl ring, which can optionally be singly or multiply substituted, wherein the substituents can be selected from the group
      halogen,
      cyano,
      R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or —C(O)—N—R⁹R¹⁰,
   wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
   a C₁-C₄ alkyl group, which is unsubstituted or can optionally be up to quintuply fluorinated,
   a C₂-C₄ alkenyl group, which is unsubstituted or can optionally be up to triply fluorinated,
   a C₂-C₄ alkynyl group, which is unsubstituted or can optionally be singly fluorinated,
   a C₃-C₆ cycloalkyl group,
   a 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which is unsubstituted or can be up to doubly substituted with fluorine, chlorine or trifluoromethyl, or
R⁹, R¹⁰ together form a 3-8-membered ring.

Likewise preferred are the compounds of the general formula I, wherein
X and Z each mean a nitrogen residue,
Y means a carbon residue —C—R⁸,
R⁸ means a hydrogen,
R¹ means an unsubstituted 5-12-membered mono or bicyclic aryl or heteroaryl ring, which is optionally singly to triply substituted,
   wherein the substituents can be selected from the group halogen,
   —C₁-C₄ alkyl, which is unsubstituted and can optionally be substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano,
   or a OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-12-membered, mono- or bicyclic aryl or heteroaryl ring, which can optionally be singly or multiply substituted,
   wherein the substituents can be selected from the group halogen, —C₁-C₄ alkyl, which is unsubstituted and can optionally be substituted, —OR⁹, —OC(O)R⁹, —S(O)ₙR⁹, wherein =0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
—R⁶, R⁷ mean a hydrogen, a C₁-C₆ alkyl group, which can be unsubstituted or optionally up to quintuply halogenated,
   an unsubstituted C₃-C₈ cycloalkyl residue,
   an unsubstituted 5-12-membered mono- or bicyclic aryl or heteroaryl ring, which can optionally be singly or multiply substituted, wherein the substituents can be selected from the group
      halogen,
      cyano,
      R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or —C(O)—N—R⁹R¹⁰,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
   an unsubstituted C₁-C₄ alkyl group, which can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein_{[S1}]
X and Y each mean a carbon residue —C—R⁸,
Z means a nitrogen residue,
R⁸ means a hydrogen or methyl group,
R¹ means an unsubstituted 5-12-membered, mono- or bicyclic aryl or heteroaryl ring, which is optionally singly to triply substituted,
   wherein the substituents can be selected from the group halogen, —C₁-C₄ alkyl, which is unsubstituted or can optionally be substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-12-membered mono- or bicyclic aryl or heteroaryl ring, which is optionally singly or multiply substituted,
   wherein the substituents can be selected from the group halogen or C₁-C₄ alkyl, which is unsubstituted or can optionally be substituted,
   —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, a C₁-C₆ alkyl group, which is unsubstituted or can optionally be up to quintuply halogenated,
   an unsubstituted C₃-C₈ cycloalkyl residue,
   an unsubstituted 5-12-membered, mono- or bicyclic aryl or heteroaryl ring, which can optionally be singly or multiply substituted, wherein the substituents can be selected from the group
      halogen,
      cyano,
      R⁹, —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CO₂—R⁹ or —C(O)—N—R⁹R¹⁰,
   wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
   an unsubstituted C₁-C₄ alkyl group, which can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X means a nitrogen residue,
Y and Z mean a carbon residue —C—R⁸,
R⁸ means a hydrogen,
R¹ means an unsubstituted 5-6-membered aryl or heteroaryl ring, which is optionally singly to triply substituted,
   wherein the substituents can be selected from the group halogen, —C₁-C₄ alkyl, which is unsubstituted or can optionally be substituted,
   —OR⁶, OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷— group,
   an unsubstituted C₁-C₆ alkyl group, which can optionally be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-6-membered aryl or heteroaryl ring, which is optionally singly or multiply substituted,
   wherein the substituents can be selected from the group
      halogen,
      —C₁-C₄ alkyl, which is unsubstituted and can optionally also be halogenated or else substituted with —OH, —CN or —CO₂H,
      —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl residue,
   an unsubstituted 5-6-membered aryl or heteroaryl ring, which can optionally be singly or multiply substituted, wherein the substituents can be selected from the group
      halogen, cyano, —R⁹, —OR⁹, —OC(O)R⁹, —S(O)ₙR⁹ wherein n=0, 1 or 2, —SO₂NHR⁹, NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group, an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
Y and Z mean a nitrogen residue,
X means a carbon residue —C—R⁸,
R⁸ means a hydrogen,
R¹ means a 5-6-membered aryl or heteroaryl ring, which is optionally singly to triply substituted,
   wherein the substituents can be selected from the group halogen, —C₁-C₄ alkyl, which is unsubstituted or can optionally be substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-6-membered aryl or heteroaryl ring, which is optionally singly or multiply substituted,
   wherein the substituents can be selected from the group
      halogen,
      —C₁-C₄ alkyl, which is unsubstituted and can optionally be halogenated or else substituted with —OH, —CN or —CO₂H,
      —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl-, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl residue, an unsubstituted 5-6-membered aryl or heteroaryl ring, which can optionally be singly or multiply substituted, wherein the substituents can be selected from the group
      halogen, cyano, —R⁹, —OR⁹, —OC(O)R⁹, —S(O)ₙR⁹ wherein n=0, 1 or 2, —SO₂NHR⁹, NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X and Z mean a nitrogen residue,
Y and Z mean a carbon residue —C—R⁸,
R⁸ means a hydrogen,
R¹ means an unsubstituted 5-6-membered aryl or heteroaryl ring, which is optionally singly to triply substituted,
   wherein the substituents can be selected from the group halogen, —C₁-C₄ alkyl, which is unsubstituted or can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶⁷ or —C(OH)R⁶R⁷,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-6-membered aryl or heteroaryl ring, which is optionally singly or multiply substituted,
   wherein the substituents can be selected from the group
      halogen,
      —C₁-C₄ alkyl, which is unsubstituted and can optionally also be halogenated or else substituted with —OH, —CN or —CO₂H,
      —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
      wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl residue,
   an unsubstituted 5-6-membered aryl or heteroaryl ring, which can optionally be singly or multiply substituted, wherein the substituents can be selected from the group
      halogen, cyano, —R⁹—OR⁹, —OC(O)R⁹, —S(O)ₙR⁹ wherein
      n=0, 1 or 2, —SO₂NHR⁹, NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
   an unsubstituted C₂-C₄ alkenyl group, which can optionally be up to triply fluorinated,
   an unsubstituted C₂-C₄ alkynyl group, which can optionally be singly fluorinated, a C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X and Y mean a carbon residue —C—R⁸,
Z means a nitrogen residue,
R⁸ means a hydrogen,
R¹ means an unsubstituted 5-6-membered aryl or heteroaryl ring, which is optionally singly to triply substituted,
   wherein the substituents can be selected from the group halogen,
      —C₁-C₄ alkyl, which is unsubstituted or can optionally be substituted,
      —OR⁶, —OC(O)R⁶, —S(O)ₙR⁶ wherein n=0, 1 or 2,
      —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁶, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be substituted,
   an unsubstituted C₃-C₁₀ cycloalkyl ring, which can optionally be substituted,
   an unsubstituted C₂-C₆ alkenyl or C₂-C₆ alkynyl group, which can optionally be substituted,
   an unsubstituted 5-6-membered aryl or heteroaryl ring, which is optionally singly or multiply substituted,
   wherein the substituents can be selected from the group halogen,
      —C₁-C₄ alkyl, which is unsubstituted and can optionally also be halogenated or else substituted with —OH, —CN or —CO₂H,
      —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl residue,
   an unsubstituted 5-6-membered aryl or heteroaryl ring, which can optionally be singly or multiply substituted, wherein the substituents can be selected from the group halogen, cyano, —R⁹—OR⁹, —OC(O)R⁹, —S(O)ₙR⁹ wherein n=0, 1 or 2, —SO₂NHR⁹, NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X means a nitrogen residue,
Y and Z mean a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
   wherein the substituents can be selected from the group halogen,
      —C₁-C₄ alkyl, which can be unsubstituted or optionally substituted,
      —OR⁶, —OC(O)R⁶, —S(O)ₙR⁶, wherein n=0, 1 or 2, —SO₂NHR⁶, —SO₂NHC(O)R⁶, NR⁶R⁶, —NHC(O)R⁶, —CN, —CO₂—R⁵, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
   an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be up to quintuply halogenated or else substituted with, —CN or —CO₂H,
   an unsubstituted C₃-C₁₀ cycloalkyl ring,
   an unsubstituted C₂-C₆ alkenyl group, which can optionally be up to triply halogenated or else substituted with —CN or —CO₂H,
   an unsubstituted C₂-C₆ alkynyl group, which can optionally be singly halogenated or else substituted with —CN or —CO₂H and,
   an unsubstituted 5-6-membered aryl or heteroaryl ring, which is optionally singly or multiply substituted,
   wherein the substituents can be selected from the group halogen,
      —C₁-C₄ alkyl, which is unsubstituted and can optionally be up to quintuply halogenated or else substituted with —OH, —CN or —CO₂H,
      —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl group, an unsubstituted 5-6-membered aryl or heteroaryl ring, which can optionally be singly or multiply substituted, wherein the substituents can be selected from the group
      halogen,
      cyano,
      R⁹—OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
      wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group, an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
Y and Z mean a nitrogen residue,
X means a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
   wherein the substituents can be selected from the group halogen, —C₁-C₄ alkyl, which can be unsubstituted or optionally substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶—CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
   an unsubstituted C₁-C₆ alkyl group, which can optionally be up to quintuply halogenated or else substituted with —CN or —CO₂H,
   an unsubstituted C₃-C₁₀ cycloalkyl ring,
   an unsubstituted C₂-C₆ alkenyl group, which can optionally be up to triply halogenated or else substituted with —CN or —CO₂H,
   an unsubstituted C₂-C₆ alkynyl group, which can optionally be singly halogenated or else substituted with —CN or —CO₂H and,
   an unsubstituted 5-6-membered aryl or heteroaryl ring, which is optionally singly or multiply substituted,
   wherein the substituents can be selected from the group
      halogen,
      C₁-C₄ alkyl, which is unsubstituted and can optionally be up to quintuply halogenated or else substituted with —OH, —CN or —CO₂H,
      OR⁹, —OC(O)R⁹—S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   an unsubstituted C₁-C₆ alkyl group, which can optionally be up to quintuply halogenated,
   an unsubstituted C₃-C₈ cycloalkyl group, an unsubstituted 5-6-membered aryl or heteroaryl ring, which can optionally be singly or multiply substituted, wherein the substituents can be selected from the group
      -halogen,
      -cyano,
      R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X and Z mean a nitrogen residue,
Y means a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
   wherein the substituents can be selected from the group halogen, —C₁-C₄ alkyl, which can be unsubstituted or optionally substituted,
   —OR⁶, —OC(O)R⁶, —S(O)ₙR⁶ wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
   an OR⁶, OC(O)R⁶, S(O)ₙR⁶, wherein n=0, 1 or 2, SO₂NHR⁶, SO₂NHC(O)R⁶, NR⁶R⁶, NHC(O)R⁶, CH₂NR⁶R⁷, CH₂NHC(O)R⁶, C(OH)R⁶R⁷, C(O)R⁶, CO₂R⁶ or C(O)NR⁶R⁷— group,
   an unsubstituted C₁-C₆ alkyl group, which can optionally be up to quintuply halogenated or else substituted with —CN or —CO₂H,
   an unsubstituted C₃-C₁₀ cycloalkyl ring,
   an unsubstituted C₂-C₆ alkenyl group, which can optionally be up to triply halogenated or else substituted with —CN or —CO₂H,
   an unsubstituted C₂-C₆ alkynyl group, which can optionally be singly halogenated or else substituted with —CN or —CO₂H, and
   an unsubstituted 5-6-membered aryl or heteroaryl ring, which is optionally singly or multiply substituted,
   wherein the substituents can be selected from the group
      halogen,
      —C₁-C₄ alkyl, which is unsubstituted and can optionally be up to quintuply halogenated or else substituted with —OH, —CN or —CO₂H,
      —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   a C₁-C₆ alkyl group, which can be unsubstituted or optionally up to quintuply halogenated,
   a C₃-C₈ cycloalkyl group,
   a 5-6-membered aryl or heteroaryl ring, which can optionally be singly or multiply substituted, wherein the substituents can be selected from the group
      halogen,
      cyano,
      an unsubstituted C₃-C₈ cycloalkyl group,
      R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group, an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

Likewise preferred are the compounds of the general formula I, wherein
X and Y mean a carbon residue —C—R⁸,
Z means a nitrogen residue,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
   wherein the substituents can be selected from the group halogen, —C₁-C₄ alkyl, which can be unsubstituted or optionally substituted,
   —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
   an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷— group,
   an unsubstituted C₁-C₆ alkyl group, which can optionally be up to quintuply halogenated or else substituted with —CN or —CO₂H,
   an unsubstituted C₃-C₁₀ cycloalkyl ring,
   an unsubstituted C₂-C₆ alkenyl group, which can optionally be up to triply halogenated or else substituted with —CN or —CO₂H,
   an unsubstituted C₂-C₆ alkynyl group, which can optionally be singly halogenated or else substituted with —CN or —CO₂H and,
   an unsubstituted 5-6-membered aryl or heteroaryl ring, which is optionally singly or multiply substituted,
   wherein the substituents can be selected from the group
      halogen,
      —C₁-C₄ alkyl, which is unsubstituted and can optionally be up to quintuply halogenated or else substituted with —OH, —CN or —CO₂H,
      —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
   a C₁-C₆ alkyl group, which is unsubstituted or can optionally be up to quintuply halogenated,
   a 5-6-membered aryl or heteroaryl ring, which can optionally be singly or multiply substituted, wherein the substituents can be selected from the group
      halogen,
      cyano,
      a C₃-C₈ cycloalkyl group,
      R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
   wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
   an unsubstituted C₂-C₄ alkenyl group, which can optionally be up to triply fluorinated,
   an unsubstituted C₂-C₄ alkynyl group, which can optionally be singly fluorinated,
   an unsubstituted C₃-C₆ cycloalkyl group,
   an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

The following compounds according to the present invention are quite especially preferred:
1-[1-(7-chloro-quinolin-4-yl)-piperidin-4-yl]-3-(4-methylsulfanyl-phenyl)-thiourea
1-[1-(7-chloro-quinolin-4-yl)-piperidin-4-yl]-3-phenyl-thiourea
1-(3-chloro-phenyl)-3-[1-(7-chloro-quinolin-4-yl)-piperidin-4-yl]-thiourea
1-(2-chloro-phenyl)-3-[1-(7-chloro-quinolin-4-yl)-piperidin-4-yl]-thiourea
1-(1-(7-chloro-quinolin-4-yl)-piperidin-4-yl)-3-(4-methoxy-phenyl)-thiourea
1-(1-(7-chloro-quinolin-4-yl)-piperidin-4-yl)-3-p-tolyl-thiourea
1-(1-(7-chloro-quinolin-4-yl)-piperidin-4-yl)-3-o-tolyl-thiourea
1-(2-bromo-phenyl)-3-(1-(7-chloro-quinolin-4-yl)-piperidin-4-yl)-thiourea
1-(3-bromo-phenyl)-3-(1-(7-chloro-quinolin-4-yl)-piperidin-4-yl)-thiourea
1-(1-(7-chloro-quinolin-4-yl)-piperidin-4-yl)-3-(3-methoxy-phenyl)-thiourea
methyl 4-(3-(1-(7-chloro-quinolin-4-yl)-piperidin-4-yl)-thioureido)-benzoate
1-(1-(7-chloro-quinolin-4-yl)-piperidin-4-yl)-3-(3-cyano-phenyl)-thiourea
1-(1-(7-chloro-quinolin-4-yl)-piperidin-4-yl)-3-(4-cyano-phenyl)-thiourea
1-(1-(7-chloro-quinolin-4-yl)-piperidin-4-yl)-3-(2-methylsulfanyl-phenyl)-thiourea
1-(1-(7-chloro-quinolin-4-yl)-piperidin-4-yl)-3-(2,3-dichloro-phenyl)-thiourea
1-(1-(7-chloro-quinolin-4-yl)-piperidin-4-yl)-3-(3-methylsulfanyl-phenyl)-thiourea
methyl 3-(3-(1-(7-chloro-quinolin-4-yl)-piperidin-4-yl)-thioureido)-benzoate
1-(1-(7-chloro-quinolin-4-yl)-piperidin-4-yl)-3-(2-trifluoromethoxy-phenyl)-thiourea
1-[1-(8-chloro-quinolin-4-yl)-piperidin-4-yl]-3-(4-methylsulfanyl-phenyl)-thiourea
1-[1-(8-chloro-quinolin-4-yl)-piperidin-4-yl]-3-phenyl-thiourea
1-(3-chloro-phenyl)-3-[1-(8-chloro-quinolin-4-yl)-piperidin-4-yl]-thiourea
1-(2-chloro-phenyl)-3-[1-(8-chloro-quinolin-4-yl)-piperidin-4-yl]-thiourea
1-(1-(8-chloro-quinolin-4-yl)-piperidin-4-yl)-3-(4-methoxy-phenyl)-thiourea
1-(1-(8-chloro-quinolin-4-yl)-piperidin-4-yl)-3-p-tolyl-thiourea
1-(1-(8-chloro-quinolin-4-yl)-piperidin-4-yl)-3-o-tolyl-thiourea
1-(2-bromo-phenyl)-3-(1-(8-chloro-quinolin-4-yl)-piperidin-4-yl)-thiourea
1-(3-bromo-phenyl)-3-(1-(8-chloro-quinolin-4-yl)-piperidin-4-yl)-thiourea
1-(1-(8-chloro-quinolin-4-yl)-piperidin-4-yl)-3-(3-methoxy-phenyl)-thiourea
methyl 4-(3-(1-(8-chloro-quinolin-4-yl)-piperidin-4-yl)-thioureido)-benzoate
1-(1-(8-chloro-quinolin-4-yl)-piperidin-4-yl)-3-(3-cyano-phenyl)-thiourea
1-(1-(8-chloro-quinolin-4-yl)-piperidin-4-yl)-3-(4-cyano-phenyl)-thiourea
1-(1-(8-chloro-quinolin-4-yl)-piperidin-4-yl)-3-(2-methylsulfanyl-phenyl)-thiourea
1-(1-(8-chloro-quinolin-4-yl)-piperidin-4-yl)-3-(2,3-dichloro-phenyl)-thiourea
1-(1-(8-chloro-quinolin-4-yl)-piperidin-4-yl)-3-(3-methylsulfanyl-phenyl)-thiourea
methyl 3-(3-(1-(8-chloro-quinolin-4-yl)-piperidin-4-yl)-thioureido)-benzoate
1-(1-(8-chloro-quinolin-4-yl)-piperidin-4-yl)-3-(2-trifluoromethoxy-phenyl)-thiourea
1-[1-(6-chloro-quinolin-4-yl)-piperidin-4-yl]-3-(4-methylsulfanyl-phenyl)-thiourea
1-[1-(6-chloro-quinolin-4-yl)-piperidin-4-yl]-3-phenyl-thiourea
1-(3-chloro-phenyl)-3-[1-(6-chloro-quinolin-4-yl)-piperidin-4-yl]-thiourea
1-(2-chloro-phenyl)-3-[1-(6-chloro-quinolin-4-yl)-piperidin-4-yl]-thiourea
1-(1-(6-chloro-quinolin-4-yl)-piperidin-4-yl)-3-(4-methoxy-phenyl)-thiourea
1-(1-(6-chloro-quinolin-4-yl)-piperidin-4-yl)-3-p-tolyl-thiourea
1-(1-(6-chloro-quinolin-4-yl)-piperidin-4-yl)-3-o-tolyl-thiourea
1-(2-bromo-phenyl)-3-(1-(6-chloro-quinolin-4-yl)-piperidin-4-yl)-thiourea
1-(3-bromo-phenyl)-3-(1-(6-chloro-quinolin-4-yl)-piperidin-4-yl)-thiourea
1-(1-(6-chloro-quinolin-4-yl)-piperidin-4-yl)-3-(3-methoxy-phenyl)-thiourea
methyl 4-(3-(1-(6-chloro-quinolin-4-yl)-piperidin-4-yl)-thioureido)-benzoate
1-(1-(6-chloro-quinolin-4-yl)-piperidin-4-yl)-3-(3-cyano-phenyl)-thiourea
1-(1-(6-chloro-quinolin-4-yl)-piperidin-4-yl)-3-(4-cyano-phenyl)-thiourea
1-(1-(6-chloro-quinolin-4-yl)-piperidin-4-yl)-3-(2-methylsulfanyl-phenyl)-thiourea
1-(1-(6-chloro-quinolin-4-yl)-piperidin-4-yl)-3-(2,3-dichloro-phenyl)-thiourea
1-(1-(6-chloro-quinolin-4-yl)-piperidin-4-yl)-3-(3-methylsulfanyl-phenyl)-thiourea
methyl 3-(3-(1-(6-chloro-quinolin-4-yl)-piperidin-4-yl)-thioureido)-benzoate
1-(1-(6-chloro-quinolin-4-yl)-piperidin-4-yl)-3-(2-trifluoromethoxy-phenyl)-thiourea
1-[1-(6-bromo-phthalazin-1-yl)-piperidin-4-yl]-3-(4-methylsulfanyl-phenyl)-thiourea
1-[1-(6-bromo-phthalazin-1-yl)-piperidin-4-yl]-3-phenyl-thiourea
1-(3-chloro-phenyl)-3-[1-(6-bromo-phthalazin-1-yl)-piperidin-4-yl]-thiourea
1-(2-chloro-phenyl)-3-[1-(6-bromo-phthalazin-1-yl)-piperidin-4-yl]-thiourea
1-(1-(6-bromo-phthalazin-1-yl)-piperidin-4-yl)-3-(4-methoxy-phenyl)-thiourea
1-(1-(6-bromo-phthalazin-1-yl)-piperidin-4-yl)-3-p-tolyl-thiourea
1-(1-(6-bromo-phthalazin-1-yl)-piperidin-4-yl)-3-o-tolyl-thiourea
1-(2-bromo-phenyl)-3-(1-(6-bromo-phthalazin-1-yl)-piperidin-4-yl)-thiourea
1-(3-bromo-phenyl)-3-(1-(6-bromo-phthalazin-1-yl)-piperidin-4-yl)-thiourea
1-(1-(6-bromo-phthalazin-1-yl)-piperidin-4-yl)-3-(3-methoxy-phenyl)-thiourea
methyl 4-(3-(1-(6-bromo-phthalazin-1-yl)-piperidin-4-yl)-thioureido)-benzoate
1-(1-(6-bromo-phthalazin-1-yl)-piperidin-4-yl)-3-(3-cyano-phenyl)-thiourea
1-(1-(6-bromo-phthalazin-1-yl)-piperidin-4-yl)-3-(4-cyano-phenyl)-thiourea
1-(1-(6-bromo-phthalazin-1-yl)-piperidin-4-yl)-3-(2-methylsulfanyl-phenyl)-thiourea
1-(1-(6-bromo-phthalazin-1-yl)-piperidin-4-yl)-3-(2,3-dichloro-phenyl)-thiourea
1-(1-(6-bromo-phthalazin-1-yl)-piperidin-4-yl)-3-(3-methylsulfanyl-phenyl)-thiourea
methyl 3-(3-(1-(6-bromo-phthalazin-1-yl)-piperidin-4-yl)-thioureido)-benzoate
1-(1-(6-bromo-phthalazin-1-yl)-piperidin-4-yl)-3-(2-trifluoromethoxy-phenyl)-thiourea
1-[1-(7-bromo-phthalazin-1-yl)-piperidin-4-yl]-3-(4-methylsulfanyl-phenyl)-thiourea
1-[1-(7-bromo-phthalazin-1-yl)-piperidin-4-yl]-3-phenyl-thiourea
1-(3-chloro-phenyl)-3-[1-(7-bromo-phthalazin-1-yl)-piperidin-4-yl]-thiourea
1-(2-chloro-phenyl)-3-[1-(7-bromo-phthalazin-1-yl)-piperidin-4-yl]-thiourea
1-(1-(7-bromo-phthalazin-1-yl)-piperidin-4-yl)-3-(4-methoxy-phenyl)-thiourea
1-(1-(7-bromo-phthalazin-1-yl)-piperidin-4-yl)-3-p-tolyl-thiourea
1-(1-(7-bromo-phthalazin-1-yl)-piperidin-4-yl)-3-o-tolyl-thiourea
1-(2-bromo-phenyl)-3-(1-(7-bromo-phthalazin-1-yl)-piperidin-4-yl)-thiourea
1-(3-bromo-phenyl)-3-(1-(7-bromo-phthalazin-1-yl)-piperidin-4-yl)-thiourea
1-(1-(7-bromo-phthalazin-1-yl)-piperidin-4-yl)-3-(3-methoxy-phenyl)-thiourea
methyl 4-(3-(1-(7-bromo-phthalazin-1-yl)-piperidin-4-yl)-thioureido)-benzoate
1-(1-(7-bromo-phthalazin-1-yl)-piperidin-4-yl)-3-(3-cyano-phenyl)-thiourea
1-(1-(7-bromo-phthalazin-1-yl)-piperidin-4-yl)-3-(4-cyano-phenyl)-thiourea
1-(1-(7-bromo-phthalazin-1-yl)-piperidin-4-yl)-3-(2-methylsulfanyl-phenyl)-thiourea
1-(1-(7-bromo-phthalazin-1-yl)-piperidin-4-yl)-3-(2,3-dichloro-phenyl)-thiourea
1-(1-(7-bromo-phthalazin-1-yl)-piperidin-4-yl)-3-(3-methylsulfanyl-phenyl)-thiourea
methyl 3-(3-(1-(7-bromo-phthalazin-1-yl)-piperidin-4-yl)-thioureido)-benzoate
1-(1-(7-bromo-phthalazin-1-yl)-piperidin-4-yl)-3-(2-trifluoromethoxy-phenyl)-thiourea
1-[1-(7-chloro-quinazolin-4-yl)-piperidin-4-yl]-3-(4-methylsulfanyl-phenyl)-thiourea
1-[1-(7-chloro-quinazolin-4-yl)-piperidin-4-yl]-3-phenyl-thiourea
1-(3-chloro-phenyl)-3-[1-(7-chloro-quinazolin-4-yl)-piperidin-4-yl]-thiourea
1-(2-chloro-phenyl)-3-[1-(7-chloro-quinazolin-4-yl)-piperidin-4-yl]-thiourea
1-(1-(7-chloro-quinazolin-4-yl)-piperidin-4-yl)-3-(4-methoxy-phenyl)-thiourea
1-(1-(7-chloro-quinazolin-4-yl)-piperidin-4-yl)-3-p-tolyl-thiourea
1-(1-(7-chloro-quinazolin-4-yl)-piperidin-4-yl)-3-o-tolyl-thiourea
1-(2-bromo-phenyl)-3-(1-(7-chloro-quinazolin-4-yl)-piperidin-4-yl)-thiourea
1-(3-bromo-phenyl)-3-(1-(7-chloro-quinazolin-4-yl)-piperidin-4-yl)-thiourea
1-(1-(7-chloro-quinazolin-4-yl)-piperidin-4-yl)-3-(3-methoxy-phenyl)-thiourea
methyl 4-(3-(1-(7-chloro-quinazolin-4-yl)-piperidin-4-yl)-thioureido)-benzoate
1-(1-(7-chloro-quinazolin-4-yl)-piperidin-4-yl)-3-(3-cyano-phenyl)-thiourea
1-(1-(7-chloro-quinazolin-4-yl)-piperidin-4-yl)-3-(4-cyano-phenyl)-thiourea
1-(1-(7-chloro-quinazolin-4-yl)-piperidin-4-yl)-3-(2-methylsulfanyl-phenyl)-thiourea
1-(1-(7-chloro-quinazolin-4-yl)-piperidin-4-yl)-3-(2,3-dichloro-phenyl)-thiourea
1-(1-(7-chloro-quinazolin-4-yl)-piperidin-4-yl)-3-(3-methylsulfanyl-phenyl)-thiourea
methyl 3-(3-(1-(7-chloro-quinazolin-4-yl)-piperidin-4-yl)-thioureido)-benzoate
1-(1-(7-chloro-q u inazolin-4-yl)-piperidin-4-yl)-3-(2-trifluoromethoxy-phenyl)-thiourea
1-(1-isoquinolin-1-yl-piperidin-4-yl)-3-(4-methylsulfanyl-phenyl)-thiourea
1-(1-isoquinolin-1-yl-piperidin-4-yl)-3-phenyl-thiourea
1-(3-chloro-phenyl)-3-(1-isoquinolin-1-yl-piperidin-4-yl)-thiourea
1-(2-chloro-phenyl)-3-(1-isoquinolin-1-yl-piperidin-4-yl)-thiourea
1-(1-isoquinolin-1-yl-piperidin-4-yl)-3-(4-methoxy-phenyl)-thiourea
1-(1-isoquinolin-1-yl-piperidin-4-yl)-3-p-tolyl-thiourea
1-(1-isoquinolin-1-yl-piperidin-4-yl)-3-o-tolyl-thiourea
1-(2-bromo-phenyl)-3-(1-isoquinolin-1-yl-piperidin-4-yl)-thiourea
1-(3-bromo-phenyl)-3-(1-isoquinolin-1-yl-piperidin-4-yl)-thiourea
1-(1-isoquinolin-1-yl-piperidin-4-yl)-3-(3-methoxy-phenyl)-thiourea
methyl 4-(3-(1-isoquinolin-1-yl-piperidin-4-yl)-thioureido)-benzoate
1-(1-isoquinolin-1-yl-piperidin-4-yl)-3-(3-cyano-phenyl)-thiourea
1-(1-isoquinolin-1-yl-piperidin-4-yl)-3-(4-cyano-phenyl)-thiourea
1-(1-isoquinolin-1-yl-piperidin-4-yl)-3-(2-methylsulfanyl-phenyl)-thiourea
1-(1-isoquinolin-1-yl-piperidin-4-yl)-3-(2,3-dichloro-phenyl)-thiourea
1-(1-isoquinolin-1-yl-piperidin-4-yl)-3-(3-methylsulfanyl-phenyl)-thiourea
methyl 3-(3-(1-isoquinolin-1-yl-piperidin-4-yl)-thioureido)-benzoate
1-(1-isoquinolin-1-yl-piperidin-4-yl)-3-(2-trifluoromethoxy-phenyl)-thiourea

An object of the present invention is the use of the compounds according to the invention for the production of drugs which contain at least one of the compounds according to formula I.

Also an object of the present invention are drugs which contain the compounds according to the invention with suitable formulation and carrier substances.

In comparison to known prostaglandin E₂ ligands, the new EP₂ agonists and antagonists are characterized by greater selectivity and stability.

An object of the present invention are drugs for the treatment and prophylaxis of diseases which include fertility disorders, infectious diseases, cancer, viral infections, cardiovascular diseases, elevated intraocular pressure, glaucoma, diseases of the skeletal system, angiogenic diseases, uterine contraction disorders, pain, neuroinflammatory diseases, immunomodulatory infections and nephrological diseases.

Fertility disorders should be understood to mean diseases which have the effect that no ovulation takes place, that nidation of a fertilized ovum does not occur and no decidualization takes place, infectious diseases to mean diseases caused by unicellular parasites, cancer to mean solid tumors and leukemia, viral infections to mean for example cytomegalus infections, hepatitis, hepatitis B and C and HIV diseases, immunomodulatory infections to mean for example avian influenza, cardiovascular diseases to mean ischemic reperfusion disease, stenosis, arteriosclerosis and restenosis, angiogenic diseases to mean for example endometriosis and fibrosis, elevated intraocular pressure to mean glaucoma, uterine contraction disorders to mean for example menstrual problems, diseases of the skeletal system to mean osteoporosis, neuroinflammatory diseases to mean multiple sclerosis, Alzheimers disease and pain, and nephrological diseases to mean glomerulonephritis.

Also an object of the present invention are drugs for the treatment and prophylaxis of the diseases listed above which contain at least one compound according to the general formula I, and drugs with suitable formulation and carrier substances.

For the use of the compounds according to the invention as drugs, these are made into the form of a pharmaceutical preparation, which in addition to the active substance contains pharmaceutical, organic or inorganic inert carrier materials suitable for enteral or parenteral administration, such as for example water, gelatin, gum arabic, lactose, starch, magnesium stearate, talc, plant oils, polyalkylene glycols and the like. The pharmaceutical preparations can be in solid form, for example as tablets, dragees, suppositories or capsules, in semisolid form, for example as ointments, creams, gels, suppositories or emulsions or in liquid form, for example as solutions, suspensions or emulsions.

If necessary, they contain additives which are for example intended to function as fillers, binders, disintegrants, lubricants, solvents, solubilizers, flavor correctors, colorant and emulsifiers. Examples of types of additive in the sense of the invention are saccharides (mono-, di-, tri-, oligo-, and/or polysaccharides), fats, waxes, oils, hydrocarbons and anionic, nonionic, cationic, natural, synthetic or semisynthetic surfactants. If necessary, they further contain additives such as preservatives, stabilizers, wetting agents or emulsifiers, and salts to modify the osmotic pressure or buffers.

These pharmaceutical preparations are also an object of the present invention.

For inhalation, it is expedient to prepare aerosol solutions.

For oral use, tablets, dragees or capsules with talc and/or hydrocarbon carriers or binders, such as for example lactose or maize or potato starch, are particularly suitable. They can also be used in liquid form, for example as a syrup, to which a sweetener is added if necessary. For the oral use of such compounds, clathrates are also suitable, and for example the clathrates with alpha, beta or gamma-cyclodextrin or also beta-hydroxypropylcyclodextrin may be mentioned.

For parenteral administration, sterile, injectable, aqueous or oily solutions are used. Particularly suitable are injection solutions or suspensions, in particular aqueous solutions of the active compounds in polyethoxylated castor oil.

For vaginal administration, for example suppositories, tampons or intrauterine devices are suitable and customary.

For intra-articular injection, suitably prepared crystal suspensions can be used.

For intramuscular injection, aqueous and oily injection solutions or suspensions and appropriate depot preparations can be used.

For rectal administration, the new compounds can be used in the form of suppositories, capsules, solutions (e.g. in the form of enemas) and ointments both for systemic and also for local therapy.

For pulmonary administration of the new compounds, these can be used in the form of aerosols and inhalation formulations.

For local use on eyes, external auditory canal, middle ear, nostrils and sinuses, the new compounds can be used as drops, ointments and tinctures in appropriate pharmaceutical preparations.

For topical application, formulations in gels, ointments, greasy ointments, creams, pastes, powders, milk and tinctures are possible. The dosage of the compounds of the general formula I in these preparations should be 0.01%-20% in order to achieve an adequate pharmacological effect.

As carrier systems, surfactant additives, such as salts of the bile acids or animal or plant phospholipids, but also mixtures thereof and liposomes or components thereof can also be used.

The dosage of the active substances can vary depending on the administration route, age and weight of the patient, nature and severity of the disease to be treated and similar factors. The treatment can be effected by single doses or by a large number of doses over a longer period. The daily dosage is 0.5-1000 mg, preferably 50-200 mg, and the dosage can be given as a single dose to be administered once or subdivided into 2 or more daily doses.

The formulations and presentations described above are also an object of the present invention.

As carrier systems, surfactant additives, such as salts of the bile acids or animal or plant phospholipids, but also mixtures thereof and liposomes or components thereof can also be used.

The administration of the compounds according to the invention can be effected by any conventional method, including oral and parenteral, for example by subcutaneous or intramuscular injections. The enteral, parenteral, vaginal and oral administrations are also an object of the present invention.

The compounds according to the invention of the general formula I bind to the EP₂ receptor and have an agonistic or antagonistic action. It is possible to determine whether there is an agonistic or antagonistic action by means of an agonism test (see Example 1.2.1. of the biological examples) or by means of an antagonism test (see Example 1.2.2. of the biological examples).

Antagonists should be understood to mean molecules which bind to their corresponding receptors and usually compete with the naturally occurring ligand of the receptor for binding to the receptor and which inhibit the initiation of the signal transduction route coupled to the receptor.

Receptor antagonists typically bind selectively to their particular receptor and not to other receptors. They normally display a higher binding affinity than the natural ligand. Although antagonists which have a higher affinity to the receptor than the natural ligand are preferred, antagonists with a lower affinity can also be used.

The antagonists preferably bind reversibly to their corresponding receptors.

The EP₂ receptor antagonist has a preferential affinity for the EP₂ receptor compared to every other EP receptor. The antagonism is measured in the presence of the natural agonist (PGE₂).

Agonists should be understood to mean molecules which bind to their corresponding receptors and usually compete with the naturally occurring ligand of the receptor for binding to the receptor and which stimulate the initiation of the signal transduction route coupled to the receptor. Agonists can also assist the binding of the natural ligand.

Receptor agonists typically bind selectively to their particular receptor and not to other receptors. They normally have a higher binding affinity than the natural ligand. Although agonists which have a higher affinity to the receptor than the natural ligand are preferred, agonists with a lower affinity can also be used.

The agonists preferably bind reversibly to their corresponding receptors.

Agonists are tested via the initiation of the signal transduction mediated by the corresponding receptor and/or their physiological action.

The compounds or low molecular weight substances which bind to a receptor are described as ligands. Their binding is usually reversible. Through the binding of a ligand to the corresponding receptor, the signal transduction route coupled to the receptor is activated or inactivated. In this manner, the ligand mediates its intracellular action. Ligands should be understood to mean agonists and antagonists of a receptor.

The substance according to Example 8 displays no inhibition in the cellular agonism test, but displays good activity (IC₅₀=3.8×10 E-6) in the antagonism test.

Also an object of the present invention is the use of the substances according to the invention as EP₂ receptor antagonists for the treatment of diseases which are caused by disorders in the signal transduction chain in which the EP₂ receptor is involved, such as for example pain and fertility disorders, as well as the use of such substances for fertility control.

The compounds according to the invention of the general formula I have a fertility promoting action. In the pre-ovulatory antral follicle, the ovum is surrounded by cumulus cells, which form a dense cell border around the ovum. After the luteinizing hormone peak (LH peak) a series of processes is activated, which results in pronounced morphological modification of this border of cumulus cells. During this, the cumulus cells form an extracellular matrix which leads to the so-called cumulus expansion (Vanderhyden et al. Dev Biol. 1990 August; 140 (2): 307-317). This cumulus expansion is an important component of the ovulatory process and the subsequent possibility of fertilization.

During cumulus expansion, prostaglandins, and here prostaglandin E₂, whose synthesis is induced by the LH peak, are of decisive importance. Prostanoid EP₂ knockout mice (Hizaki et al. Proc Natl Acad Sci. USA. 1999 Aug. 31; 96 (18): 10501-6.) display markedly decreased cumulus expansion and pronounced subfertility, which demonstrates the importance of the prostanoid EP₂ receptor for this process.

The substances according to the invention have inhibitory effects in the cumulus expansion tests.

An object of the present invention is the use of the substances according to the invention for fertility control.

While the EP₂ receptor antagonist AH 6809 only suppresses the expansion of the cumulus by ca. 30% and only at a concentration of 100-200 μM, in the presence of the substance according to Example 8 a 20% suppression of cumulus expansion can be achieved at a 10 times lower concentration. In these experiments, the test substances compete with the natural EP₂ receptor agonist PGE₂.

An object of the present invention is the use of the substances according to the invention for the inhibition of cumulus expansion, and thereby ovulation and fertilization, for contraception.

Prostaglandins play an important part in angiogenesis (Sales, Jabbour, 2003, Reproduction 126, 559-567).

Endometriosis is a chronic disease which is caused by disorders of the blood vessels. About 10% of women suffer regularly from heavy bleeding during menstruation, caused by changes in the blood vessels of the endometrium. In addition, structural differences have been observed in the blood vessels, such as for example incomplete development of the smooth muscle layer (Abberton et al., 1999, Hum. Reprod. 14, 1072-1079). Since the blood loss during menstruation is partly regulated by constriction of the blood vessels, it is obvious that the defects in the smooth musculature contribute significantly to the bleeding.

An object of the present invention is the use of the substances of the general formula I for the treatment of endometriosis.

Prostaglandins play an important part in uterine contraction; excessively strong contractions are responsible for menstrual problems (Sales, Jabbour, 2003, Reproduction 126, 559-567).

An object of the present invention is the use of the substances of the general formula I for the treatment of menstrual problems.

Prostaglandins play an important part in the onset and course of various cancer diseases (S. W. Han, Biochemical and Biophysical Research Communications 314 (2004) 1093-1099; S.-H. Chang; Cancer Research 65 (2005); 4496-9; M. D. Castellone, Science 310 (2005) 1504-1510).

An object of the present invention is the use of the substances of the general formula I for the treatment and prevention of cancer diseases.

Prostaglandins also play an important part in the processes which counteract bone loss. Hence an object of the present invention is the use of the substances according to the invention for the treatment of bone loss.

Reinold et al. (J. Clin. Invest. 115, 673-679 (2005)) describe PGE₂ receptors of the EP₂ subtype as the key signal components in inflammatory hyperalgesia. Mice which no longer carry this receptor (EP₂^{-/-}) do not feel spinal inflammatory pain. There are indications that inflammatory increased pain sensitivity can be treated by targeted modulation of EP₂ receptors.

An object of the present invention is the use of the substances according to the invention for the treatment of inflammatory hyperalgesia.

Insofar as the production of the starting compounds is not described, these are known or preparable analogously to known compounds or processes described here. It is also possible to carry out all reactions described here in parallel reactors or by means of combinatorial working techniques.

The salts are prepared in the usual manner, by treating a solution of the solution of the formula I with the equivalent quantity or an excess of a base or acid, which if necessary is in solution, and separating the precipitate or working up the solution in the usual manner.

The invention thus also relates to drugs based on the compounds of the general formula I and the usual additives or carriers.

The compounds according to the invention of the general formula I can be prepared as described in the examples. By an analogous procedure using reagents homologous to the reagents described in the examples, the further compounds of the general formula I can be obtained.

Starting from the compounds of the general formula IVa-d, the compounds according to the invention of the general formula I can be prepared by reaction with N-piperidin-4-yl-(het)arylthioureas of the general formula V by processes known to the person skilled in the art. Likewise, the compounds according to the invention of the general formula I can be prepared by conversion of compounds of the general formula IVa-d to compounds of the general formula IIIa-d and then formula IIa-d by processes known to the person skilled in the art. By an analogous procedure using reagents homologous to the reagents described in the examples, the further compounds of the general formula I can be obtained.

The residues R²-R⁵ of the compounds of the general formula I obtained in this way can be further converted to many functional groups and thus to further compounds of the general formula I by methods known to the person skilled in the art.

For example, by means of palladium(0)-catalyzed reactions by methods known to the person skilled in the art, a bromide can be replaced by an aryl or heteroaryl ring, a substituted alkene or alkyne, amine or a cyano group.

A carboxy function, cyano group or an amine functioning as R²-R⁵ can for example be converted by methods known to the person skilled in the art into esters and amides of the general formula I.

Likewise for example, after reduction to the aldehyde, ester functions or a cyano group in compounds of the general formula I can be converted by methods known to the person skilled in the art into further olefins or secondary alcohols substituted with alkyl or aryl residues. Likewise, a cyano group in compounds of the general formula I can be converted by methods known to the person skilled in the art into ketones substituted with alkyl or aryl residues, which can then be reduced to the corresponding secondary alcohols or else be converted by methods known to the person skilled in the art into tertiary alcohols substituted with alkyl or aryl residues.

The conversions of the residues R²-R⁵ in the compounds according to the invention of the general formula I just described by way of example can also be performed in the same manner by a person skilled in the art on compounds of the general formula IIa-d and IIIa-d. The compounds of the general formula IIa-d and IIIa-d thus obtained can then be converted as described into those of the formula I.

The compounds of the general formula IVa-d used for the preparation of the compounds according to the invention of the general formula I can be prepared by processes known to the person skilled in the art depending on the residues X, Y, Z and W.

In the case where X=carbon, Y=CH, Z=nitrogen and W=hydrogen this occurs by processes known to the person skilled in the art, for example starting from the phthalides of the general formula X via the 2-carboxymethylbenzoic acids of the general formula IX and alkoxymethyliden-isochroman-1,3-diones of the general formula VIII to the isoquinolinones of the general formulae VII and further to compounds thereof of the general formula IVd.

In the case where Y=CH, and X and Z each=nitrogen, this occurs by processes known to the person skilled in the art, for example starting from 2-aminobenzoic acids of the general formula XII via the quinazolinones of the general formulae XI and further to compounds thereof of the general formula IVc.

In the case where X=carbon, Y and Z each=nitrogen and W=hydrogen, this occurs by processes known to the person skilled in the art, for example starting from the phthalides of the general formula X or phthalic anhydrides of the general formula XVII via the phthalazine of the general formulae XIII to those of the general formula IVb. In the case where W=C₁-C₃ alkyl, this occurs starting from the phthalic anhydrides of the general formula XVII via the 3-hydroxy-3-alkyl-3H-isobenzofuran-1-ones of the general formula XV or the 3-alkyliden-3H-isobenzofuran-1-ones of the general formula XVI to the phthalazines of the general formulae XIII and further to those of the general formula IVb.

In the case where Y and Z each=CH and X=nitrogen, this occurs by processes known to the person skilled in the art, for example starting from anilines of the general formula XXII via the compounds of the general formula XXI and the 3-carboxyquinolines of the general formula XX to those of the general formula XIX. These are converted by processes known to the person skilled in the art to quinolines of the general formulae XVIII and further to compounds thereof of the general formula IVa.

The N-piperidin-4-yl-(het)arylthioureas of the general formula V used for the preparation of the compounds according to the invention of the general formula I can be prepared by methods known to the person skilled in the art starting from tert.-butyl 4-amino-piperidin-1-carboxylate via the thioureas of the general formula VI.

### Commonly Used Abbreviations:

### DMF N,N-dimethylformamide

equiv. equivalents
DMAP 4-dimethylaminopyridine

### General Synthetic Procedure

The appropriate amine IIa-d (1 equiv.) is taken and treated with DMF (2 ml/mmol). To this are successively added the appropriate isothiocyanate (1.1 equiv.) in DMF (2 ml/mmol) and DMAP (1.1 equiv.) in DMF (1 mL/mmol) and the mixture is stirred for 12 hrs at 100° C. bath temperature. For the workup, this is cooled, treated with methanol (5 mL/mmol) and concentrated in vacuo. The residue is purified by preparative HPLC-MS and the products are characterized by analytical HPLC-MS [method 1: column HiBar RT 125-4 (125×4.5 mm, RP18e, 5 μm), gradient 5-95% acetonitrile (0.1% trifluoroacetic acid) in water (0.1% trifluoroacetic acid) (10 min.), flow rate 1.0 mL/min, MS ES+; method 2: column Aquity HPLC BEH (2,1×50 mm C18 1.7 um), gradient: start 98% A (water+0.05% formic acid)+2% B (acetonitrile+0.05% formic acid), in 1.7 min to 10% A+90% B, 0.2 min isocratic, flow rate: 1.3 ml/min, MS ES+].

| | | | MW | MW | RT [min., |
|---|---|---|---|---|---|
| Example | Structure | Name | calc. | ES+ | (method)] |
| 1 | 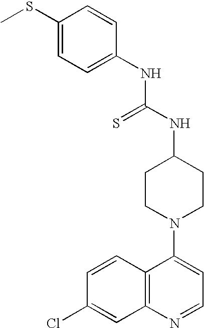 | 1-[1-(7-chloro-quinolin-4-yl)-piperidin-4-yl]-3-(4-methylsulfanyl-phenyl)-thiourea | 443.036 | 443 | 0.94 (2) |
| 2 | 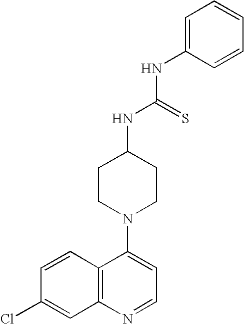 | 1-[1-(7-chloro-quinolin-4-yl)-piperidin-4-yl]-3-phenyl-thiourea | 396.943 | 397 | 0.86 (2) |
| 3 | 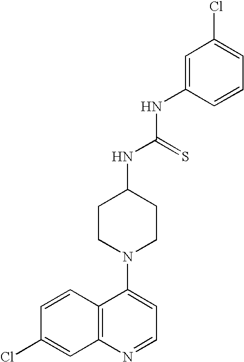 | 1-(3-chloro-phenyl)-3-[1-(7-chloro-quinolin-4-yl)-piperidin-4-yl]-thiourea | 431.388 | 431 | 7.62 (1) |
| 4 | 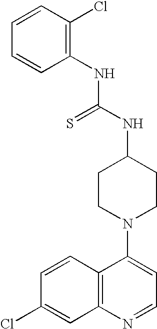 | 1-(2-chloro-phenyl)-3-[1-(7-chloro-quinolin-4-yl)-piperidin-4-yl]-thiourea | 431.388 | 431 | 7.24 (1) |
| 5 | 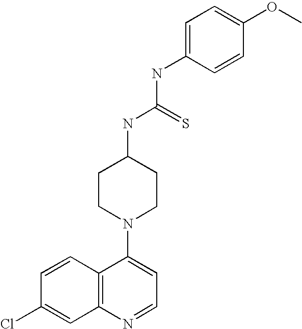 | 1-(1-(7-chloro-quinolin-4-yl)-piperidin-4-yl)-3-(4-methoxy-phenyl)-thiourea | 426.97 | 427 | 6.90 (1) |
| 6 | 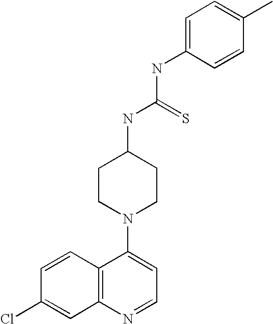 | 1-(1-(7-chloro-quinolin-4-yl)-piperidin-4-yl)-3-p-tolyl-thiourea | 410.97 | 411 | 7.24 (1) |
| 7 | 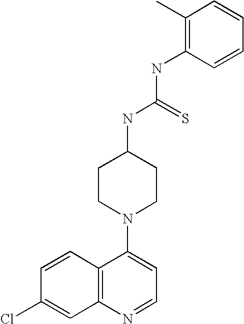 | 1-(1-(7-chloro-quinolin-4-yl)-piperidin-4-yl)-3-o-tolyl-thiourea | 410.97 | 411 | 6.99 (1) |
| 8 | 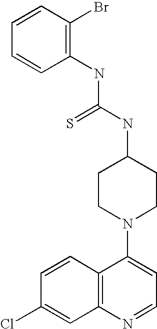 | 1-(2-bromo-phenyl)-3-(1-(7-chloro-quinolin-4-yl)-piperidin-4-yl)-thiourea | 475.839 | 477 | 7.19 (1) |
| 9 | 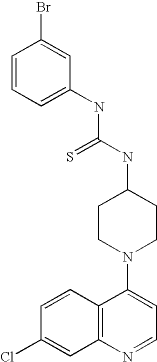 | 1-(3-bromo-phenyl)-3-(1-(7-chloro-quinolin-4-yl)-piperidin-4-yl)-thiourea | 475.839 | 477 | 7.70 (1) |
| 10 | 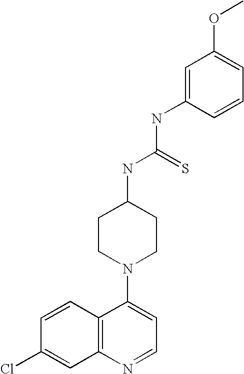 | 1-(1-(7-chloro-quinolin-4-yl)-piperidin-4-yl)-3-(3-methoxy-phenyl)-thiourea | 426.97 | 427 | 7.12 (1) |
| 11 | 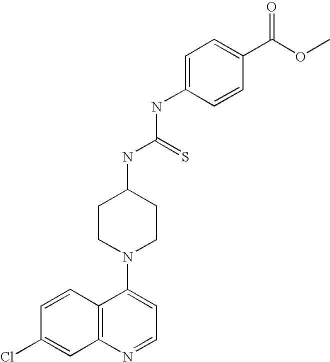 | methyl 4-(3-(1-(7-chloro-quinolin-4-yl)-piperidin-4-yl)-thioureido)-benzoate | 454.98 | 455 | 7.22 (1) |
| 12 | 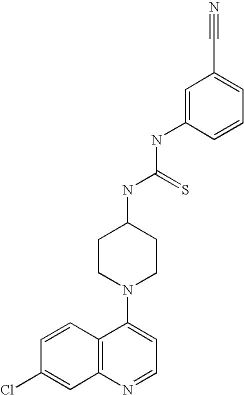 | 1-(1-(7-chloro-quinolin-4-yl)-piperidin-4-yl)-3-(3-cyano-phenyl)-thiourea | 421.953 | 422 | 7.09 (1) |
| 13 | 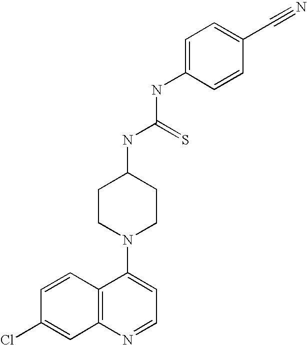 | 1-(1-(7-chloro-quinolin-4-yl)-piperidin-4-yl)-3-(4-cyano-phenyl)-thiourea | 421.953 | 422 | 7.16 (1) |
| 14 | 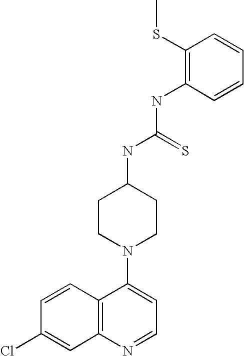 | 1-(1-(7-chloro-quinolin-4-yl)-piperidin-4-yl)-3-(2-methylsulfanyl-phenyl)-thiourea | 443.036 | 443 | 7.18 (1) |
| 15 | 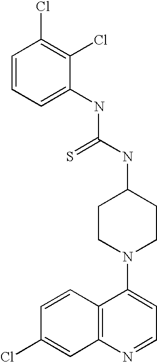 | 1-(1-(7-chloro-quinolin-4-yl)-piperidin-4-yl)-3-(2,3-dichloro-phenyl)-thiourea | 465.833 | 467 | 7.76 (1) |
| 16 | 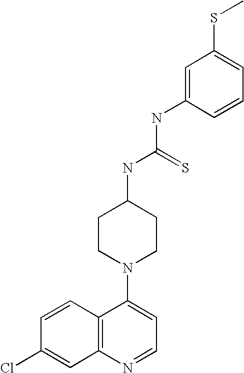 | 1-(1-(7-chloro-quinolin-4-yl)-piperidin-4-yl)-3-(3-methylsulfanyl-phenyl)-thiourea | 443.036 | 443 | 7.47 (1) |
| 17 | 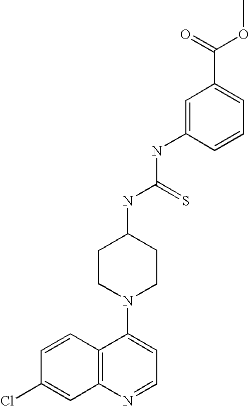 | methyl 3-(3-(1-(7-chloro-quinolin-4-yl)-piperidin-4-yl)-thioureido)-benzoate | 454.98 | 455 | 7.14 (1) |
| 18 | 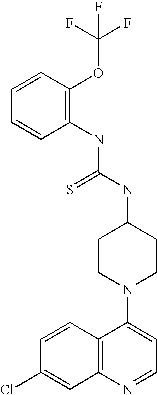 | 1-(1-(7-chloro-quinolin-4-yl)-piperidin-4-yl)-3-(2-trifluoro-methoxy-phenyl)-thiourea | 480.941 | 481 | 7.70 (1) |

Synthesis Schemes

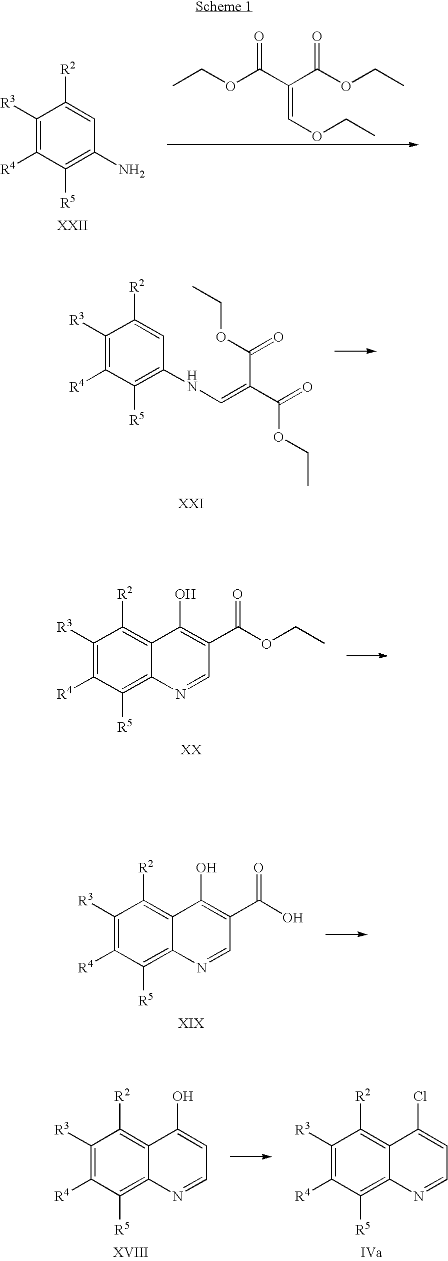

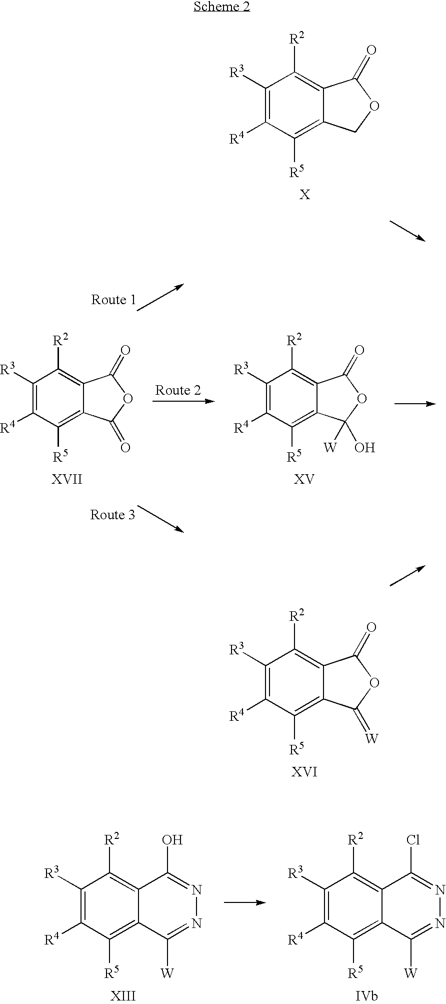

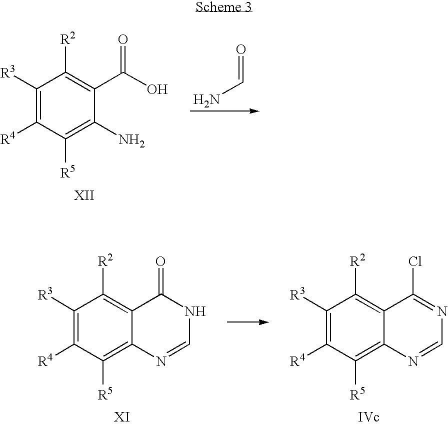

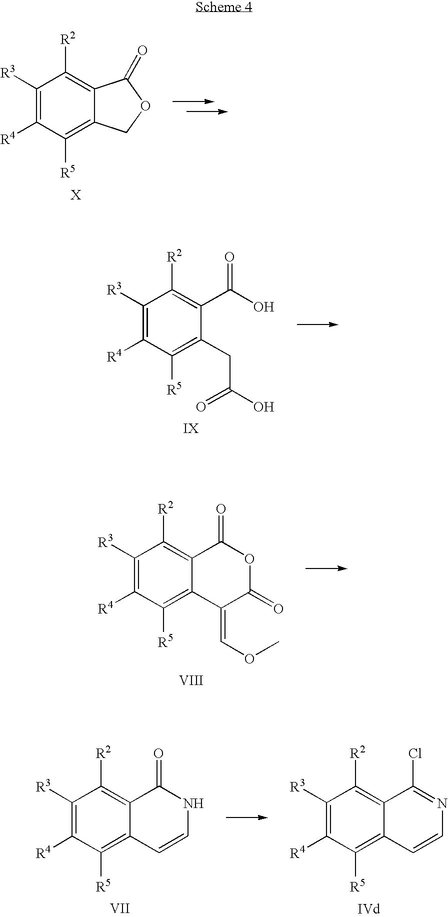

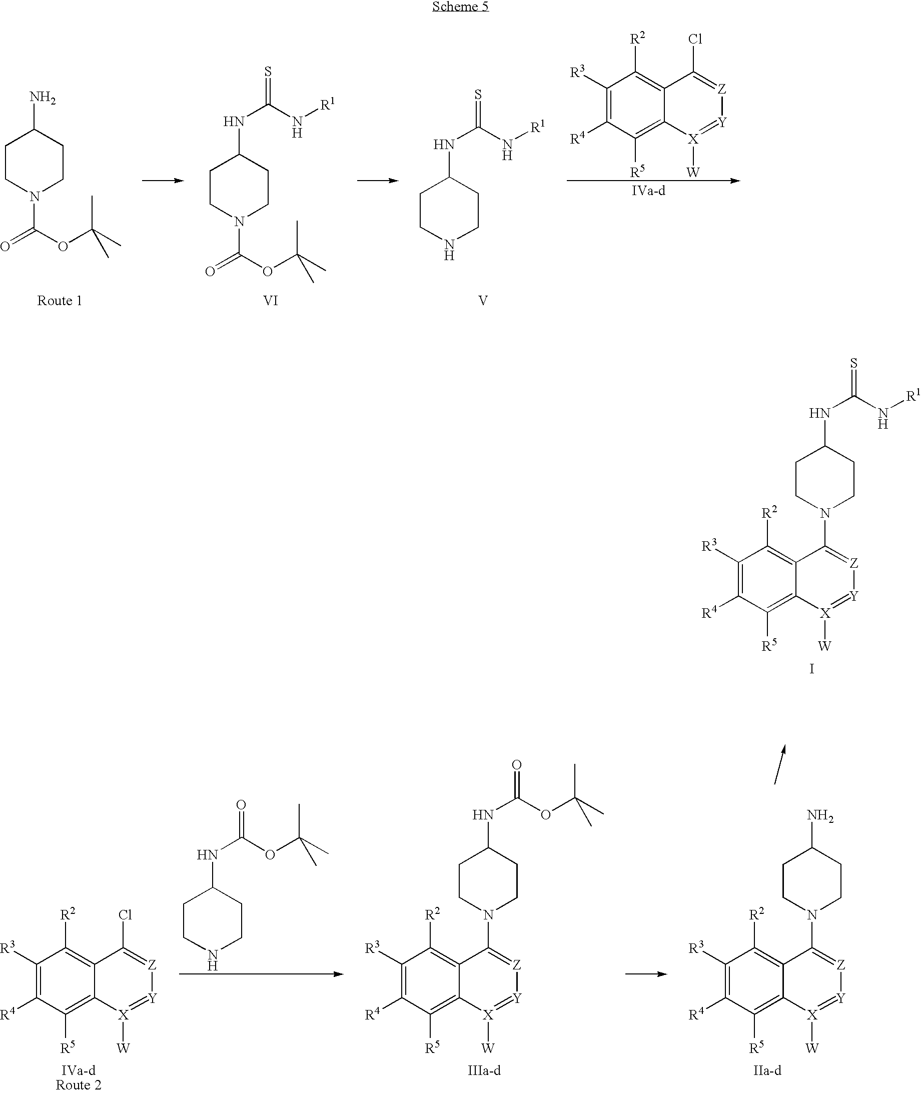

### BIOLOGICAL EXAMPLES

### 1. Detection of Antagonism of the Human Prostaglandin E₂ (Subtype EP₂ Receptor Signal

### 1.1 Detection Principle

The binding of PGE₂ to the EP₂ subtype of the human PGE₂ receptor induces the activation of membrane adenylate cyclases and leads to the formation of cAMP. In the presence of the phosphodiesterase inhibitor IBMX, the cAMP accumulated through this stimulation and released by cell lysis is used in a competitive detection process. In this test, the cAMP in the lysate competes with cAMP-XL665 for the binding of an Eu cryptate-labeled anti-cAMP antibody. Here in the absence of cellular cAMP a maximal signal is produced, which is due to the coupling of this antibody to the cAMP-XL665 molecule. As a result, after excitation at 337 nm, a long-lasting emission signal at 665 nm (and at 620 nm) based on FRET (fluorescence resonance energy transfer) is produced. Both signals are measured in a suitable measuring instrument with time delay, i.e. after the background fluorescence has died away. Any increase in the low FRET signal (measured as wavelength ratio change=emission₆₆₅ₙₘ/emission₆₂₀ₙₘ*10000) caused by administration of prostaglandin E₂ demonstrates the action of antagonists.

### 1.2. Detection Procedure

### 1.2.1. Test for Antagonism (Data Per Well of a 384-Well Plate):

The substance solutions (0.75 μl) which contain 30% DMSO are placed in a test plate and are dissolved in 16 μl of a KRSB+IBMX stimulation solution (1× cancer Ringer bicarbonate buffer; Sigma-Aldrich # K-4002; including 750 μM 3-isobutyl-1-methylxanthine Sigma-Aldrich # 1-7018), then 15 μl of this are transferred into a media-free cell culture plate which has been washed with KRSB shortly beforehand.

After a 30-minute preincubation at room temperature (RT), 5 μl of a 4×PGE₂ solution (11 nM) are added and incubated in the presence of the agonist for a further 60 mins at RT (volume: ˜20 μl), before the reaction is then stopped by addition of 5 μl of lysis buffer and the mixture is incubated for a further 20 mins at RT (volume: ˜25 μl). The cell lysate is then transferred onto an assay plate and assayed in accordance with the manufacturer's instructions (cyclic AMP kit Cisbio International # 62AMPPEC).

### 1.2.2. Test for Agonism (Data Per Well of a 384-Well Plate):

The substance solutions (0.75 μl) placed in a test plate and 30% DMSO are dissolved in 16 μl of a KRSB+IBMX stimulation solution (1× cancer Ringer bicarbonate buffer; Sigma-Aldrich # K-4002; including 750 μM 3-isobutyl-1-methylxanthine Sigma-Aldrich # 1-7018), then 15 μl of this are transferred into a media-free cell culture plate which has been washed with KRSB shortly beforehand.

After a 60-minute preincubation at room temperature (RT; volume: ˜15 μl), the reaction is then stopped by addition of 5 μl of lysis buffer and the mixture is incubated for a further 20 mins at RT (volume: ˜20 μl). The cell lysate is then transferred onto an assay plate and assayed in accordance with the manufacturer's instructions (cyclic AMP kit Cisbio International # 62AMPPEC).

### 2. The EP₂ Subtype of the PGE₂ Receptor and Pre-Ovulatory Cumulus Expansion

### 2.1. Background:

In the pre-ovulatory antral follicle, the ovum is surrounded by cumulus cells, which form a dense cell border around the ovum. After the LH peak (luteinizing hormone), a series of processes is activated, which results in pronounced morphological modification of this cell border of cumulus cells. During this, the cumulus cells form an extracellular matrix which leads to the so-called cumulus expansion (Vanderhyden et al. Dev Biol. 1990 August; 140 (2): 307-317). This cumulus expansion is an important component of the ovulatory process and the subsequent possibility of fertilization.

During cumulus expansion, prostaglandins, and here prostaglandin E₂, whose synthesis is induced by the LH peak, are of decisive importance. Prostanoid EP₂ knockout mice (Hizaki et al. Proc Natl Acad Sci USA. 1999 Aug. 31; 96 (18): 10501-6.) display markedly decreased cumulus expansion and pronounced subfertility, which demonstrates the importance of the prostanoid EP₂ receptor for this process.

### 2.2. Cumulus Expansion Test In Vitro

In immature female mice (strain: CD1 (ICR) von Charles River), folliculogenesis is induced at an age of 14-18 days by a single administration (intraperitoneal) of 10 I.U. of PMSG (Pregnant Mare Serum Gonadotropin; Sigma G-4877, Lot 68H0909). 47-50 hours after the injection, the ovaries are removed and the cumulus-ovum complexes removed. At this stage, the cumulus complex is not yet expanded.

The cumulus-ovum complexes are now incubated for 20-24 hours with prostaglandin E₂ (PGE₂) (1 μM), vehicle control (ethanol) or test substances. Medium: alpha-MEM medium with 0.1 mM IBMX, pyruvate (0.23 mM) glutamine (2 mM), pen/strep 100 IU/ml pen. and 100 μg/ml strep.) and HSA (8 mg/ml)). The cumulus expansion is then assessed by classification into four stages (after Vanderhyden et al. Dev Biol. 1990 August; 140(2):307-317).

TABLE 1
Example of the biological activity of the compounds according to the
invention (measured by cAMP antagonism test):

| Substance | |
|---|---|
| according to | Antagonism |
| example | [IC₅₀, μM] |
| 8 | 3.8 |
| 1 | 2.7 |
| 14 | 3.4 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 06 090 158.4, filed Sep. 7, 2006, and U.S. Provisional Application Ser. No. 60/842,680 filed Sep. 7, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I 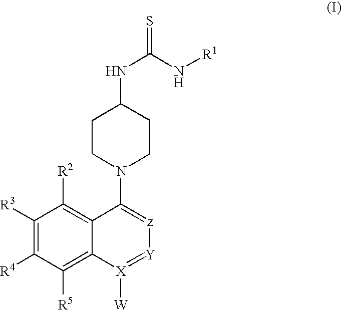
wherein
X, Y, Z independently of each other mean a nitrogen residue or a carbon residue —C—R⁸, wherein R⁸ can be hydrogen or a C₁-C₄ alkyl residue,
under the condition that at least one, but at most 2 of the groups X, Y and Z is a nitrogen residue,
R¹ means a 5-12-membered, mono- or bicyclic aryl or heteroaryl ring, which can be unsubstituted or optionally singly to triply substituted,
R²-R⁵ independently of each other mean a hydrogen, halogen, cyano,
or a OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which can be unsubstituted or optionally substituted
a C₃-C₁₀ cycloalkyl ring, which can be unsubstituted or optionally substituted,
a C₂-C₆ alkenyl or C₂-C₆ alkynyl group, which can be unsubstituted or optionally substituted,
a 5-12-membered, mono- or bicyclic aryl or heteroaryl ring, which can be unsubstituted or optionally substituted,
R⁶, R⁷ mean a hydrogen, a C₁-C₆ alkyl, a C₃-C₁₀ cycloalkyl ring, a 5-12-membered mono- or bicyclic aryl or heteroaryl ring, wherein the alkyl, cycloalkyl and (het)aryl groups can be unsubstituted or optionally substituted or
R⁶, R⁷ together form a 3-8-membered ring,
and isomers and salts thereof and cyclodextrin clathrates thereof,
which can overcome known disadvantages and achieve better selectivity for the EP₂ receptor and thus better efficacy and longer duration of action.

2. The compounds as claimed in claim 1, wherein
X means a nitrogen residue,
Y, Z mean a carbon residue —C—R⁸,
R⁸ means a hydrogen or a C₁-C₄ alkyl residue, R¹ means a 5-12-membered, mono- or bicyclic, aryl or heteroaryl ring, which can be unsubstituted and optionally singly to triply substituted,
R²-R⁵ independently of each other mean a hydrogen, halogen, cyano,
or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is unsubstituted or can optionally be substituted,
a C₃-C₁₀ cycloalkyl ring, which is unsubstituted or can optionally be substituted,
a C₂-C₆ alkenyl- or C₂-C₆ alkynyl group, which is unsubstituted or can optionally be substituted,
a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered mono- or bicyclic aryl or heteroaryl ring, wherein the alkyl, cycloalkyl and (het)aryl groups are unsubstituted or can optionally be substituted or
R⁶, R⁷ together form a 3-8-membered ring.

3. The compounds as claimed in claim 1, wherein
Y and Z mean a nitrogen residue,
X means a carbon residue —C—R⁸,
R⁸ means a hydrogen or a C₁-C₄ alkyl residue,
R¹ means a 5-12-membered mono or bicyclic aryl or heteroaryl ring, which is unsubstituted or can optionally be singly to triply substituted,
R²-R⁵ independently of each other mean a hydrogen, halogen, cyano,
or a OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is unsubstituted or can optionally be substituted,
a C₃-C₁₀ cycloalkyl ring, which is unsubstituted or can optionally be substituted,
a C₂-C₆ alkenyl or C₂-C₆ alkynyl group, which is unsubstituted or can optionally be substituted,
a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered mono- or bicyclic aryl or heteroaryl ring, wherein the alkyl, cycloalkyl and (het)aryl groups are unsubstituted or can optionally be substituted or
R⁶, R⁷ together form a 3-8-membered ring.

4. The compounds as claimed in claim 1, wherein
X and Z each mean a nitrogen residue,
Y means a carbon residue —C—R⁸,
R⁸ means a hydrogen or a C₁-C₄ alkyl residue,
R¹ means a 5-12-membered mono- or bicyclic aryl or heteroaryl ring, which is unsubstituted and can optionally be singly to triply substituted,
R²-R⁵ independently of each other mean a hydrogen, halogen, cyano,
or a OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is unsubstituted or can optionally be substituted,
a C₃-C₁₀ cycloalkyl ring, which is unsubstituted or can optionally be substituted,
a C₂-C₆ alkenyl or C₂-C₆ alkynyl group, which is unsubstituted or can optionally be substituted,
a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered, mono- or bicyclic aryl or heteroaryl ring, wherein the alkyl, cycloalkyl and (het)aryl groups are unsubstituted or can optionally be substituted or
R⁶, R⁷ together form a 3-8-membered ring.

5. The compounds as claimed in claim 1, wherein
X and Y each mean a carbon residue —C—R⁸,
Z means a nitrogen residue,
R⁸ means a hydrogen or a C₁-C₄ alkyl residue,
R¹ means a 5-12-membered or mono- or bicyclic aryl or heteroaryl ring, which is unsubstituted or can optionally be singly to triply substituted,
R²-R⁵ independently of each other mean a hydrogen, halogen, cyano,
or a OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is unsubstituted or can optionally be substituted,
a C₃-C₁₀ cycloalkyl ring, which is unsubstituted or can optionally be substituted,
a C₂-C₆ alkenyl or C₂-C₆ alkynyl group, which is unsubstituted or can optionally be substituted,
a 5-12-membered mono- or bicyclic aryl or heteroaryl ring, which is unsubstituted or can optionally be substituted,
R⁶, R⁷ mean a hydrogen, a C₁-C₆ alkyl, a C₃-C₁₀ cycloalkyl ring, a 5-12-membered, mono- or bicyclic aryl or heteroaryl ring, wherein the alkyl, cycloalkyl and (het)aryl groups are unsubstituted or can optionally be substituted or
R⁶, R⁷ together form a 3-8-membered ring.

6. The compounds as claimed in claim 1, wherein
X means a nitrogen residue,
Y, Z mean a carbon residue —C—R⁸,
R⁸ means a hydrogen,
R¹ means an unsubstituted 5-12-membered mono- or bicyclic aryl or heteroaryl ring, which is optionally singly to triply substituted,
wherein the substituents can be selected from the group halogen,
—C₁-C₄ alkyl, which is unsubstituted and can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano,
or a OR⁶, OC(O)R⁶, S(O)NR⁶ wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is optionally unsubstituted or substituted,
a C₃-C₁₀ cycloalkyl ring, which is optionally unsubstituted or substituted,
a C₂-C₆ alkenyl or C₂-C₆ alkynyl group, which is optionally substituted or unsubstituted,
a 5-12-membered mono- or bicyclic aryl or heteroaryl ring, which is optionally singly or multiply substituted,
wherein the substituents can be selected from the group
halogen,
—C₁-C₄ alkyl, which is unsubstituted and can optionally be substituted,
—OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, a C₁-C₆ alkyl group, which is unsubstituted or can optionally be up to quintuply halogenated,
a C₃-C₈ cycloalkyl residue,
a 5-12-membered, mono or bicyclic aryl or heteroaryl ring, which is optionally singly or multiply substituted,
wherein the substituents can be selected from the group
halogen,
cyano,
R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or C(O)—N—R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
a C₁-C₄ alkyl group, which is unsubstituted or can optionally be up to quintuply fluorinated,
a C₂-C₄ alkenyl group, which is unsubstituted or can optionally be up to triply fluorinated,
a C₂-C₄ alkynyl group, which is unsubstituted or can optionally be singly fluorinated,
a C₃-C₆ cycloalkyl group,
a 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which is unsubstituted or can be up to doubly substituted with fluorine, chlorine or trifluoromethyl, or
R⁹, R¹⁰ together form a 3-8-membered ring.

7. The compounds as claimed in claim 1, wherein
Y and Z mean a nitrogen residue,
X means a carbon residue —C—R⁸,
R⁸ means a hydrogen or a methyl group,
R¹ means a 5-12-membered mono- or bicyclic aryl or heteroaryl ring, which is optionally singly to triply substituted,
wherein the substituents can be selected from the group halogen, —C₁-C₄ alkyl, which is unsubstituted and can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano,
or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
a C₁-C₆ alkyl group, which is unsubstituted or can optionally be substituted,
a C₃-C₁₀ cycloalkyl ring, which is unsubstituted or can optionally be substituted,
a C₂-C₆ alkenyl or C₂-C₆ alkynyl group, which is unsubstituted or can optionally be substituted,
an unsubstituted 5-12-membered mono- or bicyclic aryl or heteroaryl ring, which can optionally be singly or multiply substituted,
wherein the substituents can be selected from the group halogen,
—C₁-C₄ alkyl, which is unsubstituted and can optionally be substituted, —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, an unsubstituted C₁-C₆ alkyl group, which can optionally be up to quintuply halogenated,
a C₃-C₈ cycloalkyl residue,
an unsubstituted 5-12-membered, mono or bicyclic aryl or heteroaryl ring, which can optionally be singly or multiply substituted, wherein the substituents can be selected from the group
halogen,
cyano,
R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or —C(O)—N—R⁹R¹⁰,
wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
a C₁-C₄ alkyl group, which is unsubstituted or can optionally be up to quintuply fluorinated,
a C₂-C₄ alkenyl group, which is unsubstituted or can optionally be up to triply fluorinated,
a C₂-C₄ alkynyl group, which is unsubstituted or can optionally be singly fluorinated,
a C₃-C₆ cycloalkyl group,
a 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which is unsubstituted or can be up to doubly substituted with fluorine, chlorine or trifluoromethyl, or
R⁹, R¹⁰ together form a 3-8-membered ring.

8. The compounds as claimed in claim 1, wherein
X and Z each mean a nitrogen residue,
Y means a carbon residue —C—R⁸,
R⁸ means a hydrogen,
R¹ means an unsubstituted 5-12-membered mono or bicyclic aryl or heteroaryl ring, which is optionally singly to triply substituted,
wherein the substituents can be selected from the group halogen, —C₁-C₄ alkyl, which is unsubstituted and can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-12-membered mono or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano,
or a OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can be substituted,
an unsubstituted C₃-C₁₀-cycloalkyl ring, which can optionally be substituted,
an unsubstituted C₂-C₆ alkenyl or C₂-C₆ alkynyl group, which can optionally be substituted,
an unsubstituted 5-12-membered, mono- or bicyclic aryl or heteroaryl ring, which can optionally be singly or multiply substituted, wherein the substituents can be selected from the group halogen, —C₁-C₄ alkyl, which is unsubstituted and can optionally be substituted, —OR⁹, —OC(O)R⁹, —S(O)ₙR⁹, wherein =0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, a C₁-C₆ alkyl group, which can be unsubstituted or optionally up to quintuply halogenated,
an unsubstituted C₃-C₈ cycloalkyl residue,
an unsubstituted 5-12-membered mono- or bicyclic aryl or heteroaryl ring, which can optionally be singly or multiply substituted, wherein the substituents can be selected from the group
halogen,
cyano,
R⁹, —OR⁹, —OC(O)R⁹, —S(O)ₙR⁹, wherein n=0, 1 or 2, —SO₂NHR⁹, NR⁹R¹⁰, —NHC(O)R⁹, —CO₂—R⁹ or —C(O)—N—R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
an unsubstituted C₁-C₄ alkyl group, which can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

9. The compounds as claimed in claim 1, wherein
X and Y each mean a carbon residue —C—R⁸,
Z means a nitrogen residue,
R⁸ means a hydrogen or methyl group, R¹ means an unsubstituted 5-12-membered, mono- or bicyclic aryl or heteroaryl ring, which is optionally singly to triply substituted,
wherein the substituents can be selected from the group halogen, —C₁-C₄ alkyl, which is unsubstituted or can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R² means a hydrogen,
R³-R⁵ independently of each other mean a hydrogen, halogen, cyano, or
an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be substituted,
an unsubstituted C₃-C₁₀ cycloalkyl ring, which can optionally be substituted,
an unsubstituted C₂-C₆ alkenyl or C₂-C₆ alkynyl group, which can optionally be substituted,
an unsubstituted 5-12-membered mono- or bicyclic aryl or heteroaryl ring, which is optionally singly or multiply substituted,
wherein the substituents can be selected from the group halogen, —C₁-C₄ alkyl, which is unsubstituted or can optionally be substituted, —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group,
R⁶, R⁷ mean a hydrogen, a C₁-C₆ alkyl group, which is unsubstituted or can optionally be up to quintuply halogenated,
an unsubstituted C₃-C₈ cycloalkyl residue,
an unsubstituted 5-12-membered, mono- or bicyclic aryl or heteroaryl ring, which can optionally be singly or multiply substituted, wherein the substituents can be selected from the group
halogen,
cyano,
R⁹, —OR⁹, —OC(O)R⁹, —S(O)ₙR⁹ wherein n=0, 1 or 2, —SO₂NHR⁹, —SO₂NHC(O)R⁹, NR⁹R¹⁰, —NHC(O)R⁹, —CO₂—R⁹ or —C(O)—N—R⁹R¹⁰,
wherein the 5-12-membered mono- or bicyclic aryl or heteroaryl ring can for example, but not exclusively, be a naphthyl, quinolinyl, isoquinolinyl, phthalazinyl, quinazolinyl, quinoxalinyl, cinnolinyl, benzothiophenyl, 1,3-benzodioxolyl, 2,1,3-benzothiadiazolyl, phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl, tetrazolyl, indolyl, benzofuranyl or benzimidazolyl group, or
R⁶, R⁷ together form a 3-8-membered ring,
R⁹, R¹⁰ independently of each other mean hydrogen,
an unsubstituted C₁-C₄ alkyl group, which can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

10. The compounds as claimed in claim 1, wherein
X means a nitrogen residue,
Y and Z mean a carbon residue —C—R⁸,
R⁸ means a hydrogen,
R¹ means an unsubstituted 5-6-membered aryl or heteroaryl ring, which is optionally singly to triply substituted,
wherein the substituents can be selected from the group halogen, —C₁-C₄ alkyl, which is unsubstituted or can optionally be substituted, —OR⁶, —OC(O)R⁶, —S(O)ᵣR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷— group,
an unsubstituted C₁-C₆ alkyl group, which can optionally be substituted,
an unsubstituted C₃-C₁₀ cycloalkyl ring, which can optionally be substituted,
an unsubstituted C₂-C₆ alkenyl or C₂-C₆ alkynyl group, which can optionally be substituted,
an unsubstituted 5-6-membered aryl or heteroaryl ring, which is optionally singly or multiply substituted,
wherein the substituents can be selected from the group halogen,
—C₁-C₄ alkyl, which is unsubstituted and can also optionally be halogenated or else substituted with
—OH, —CN or —CO₂H,
—OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl residue,
an unsubstituted 5-6-membered aryl or heteroaryl ring, which can optionally be singly or multiply substituted, wherein the substituents can be selected from the group
halogen, cyano, —R⁹, —OR⁹, —OC(O)R⁹, —S(O)ₙR⁹ wherein
n=0, 1 or 2, —SO₂NHR⁹, NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

11. The compounds as claimed in claim 1, wherein
Y and Z mean a nitrogen residue,
X means a carbon residue —C—R⁸,
R⁸ means a hydrogen,
R¹ means a 5-6-membered aryl or heteroaryl ring, which is optionally singly to triply substituted,
wherein the substituents can be selected from the group halogen, —C₁-C₄ alkyl, which is unsubstituted or can optionally be substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be substituted,
an unsubstituted C₃-C₁₀ cycloalkyl ring, which can optionally be substituted,
an unsubstituted C₂-C₆ alkenyl or C₂-C₆ alkynyl group, which can optionally be substituted,
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally be halogenated or else substituted with —OH, —CN or —CO₂H,
—OR⁹, —OC(O)R⁹, —S(O)ₙR⁹ wherein n=0, 1 or 2,
—SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl residue,
an unsubstituted 5-6-membered aryl or heteroaryl ring, which can optionally be singly or multiply substituted, wherein the substituents can be selected from the group
halogen, cyano, —R⁹—OR⁹, —OC(O)R⁹, —S(O)ₙR⁹ wherein
n=0, 1 or 2, —SO₂NHR⁹, NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

12. The compounds as claimed in claim 1, wherein
X and Z mean a nitrogen residue,
Y and Z mean a carbon residue —C—R⁸,
R⁸ means a hydrogen,
R¹ means an unsubstituted 5-6-membered aryl or heteroaryl ring, which is optionally singly to triply substituted,
wherein the substituents can be selected from the group halogen, —C₁-C₄ alkyl, which is unsubstituted or can optionally be substituted,
—OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be substituted,
an unsubstituted C₃-C₁₀ cycloalkyl ring, which can optionally be substituted,
an unsubstituted C₂-C₆ alkenyl or C₂-C₆ alkynyl group, which can optionally be substituted,
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally also be halogenated or else substituted with —OH,
—CN or —CO₂H,
—OR⁹, —OC(O)R⁹, —S(O)ₙR⁹ wherein n=0, 1 or 2,
—SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl residue,
an unsubstituted 5-6-membered aryl or heteroaryl ring, which can optionally be singly or multiply substituted, wherein the substituents can be selected from the group
halogen, cyano, —R⁹—OR⁹, —OC(O)R⁹, —S(O)ₙR⁹ wherein
n=0, 1 or 2, —SO₂NHR⁹, NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
an unsubstituted C₂-C₄ alkenyl group, which can optionally be up to triply fluorinated,
an unsubstituted C₂-C₄ alkynyl group, which can optionally be singly fluorinated, a C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

13. The compounds as claimed in claim 1, wherein
X and Y mean a carbon residue —C—R⁸,
Z means a nitrogen residue,
R⁸ means a hydrogen,
R¹ means an unsubstituted 5-6-membered aryl or heteroaryl ring, which is optionally singly to triply substituted,
wherein the substituents can be selected from the group
halogen,
—C₁-C₄ alkyl, which is unsubstituted or can optionally be substituted, —OR⁶, —OC(O)R⁶, —S(O)ₙR⁶, wherein n=0, 1 or 2,
—SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R² means a hydrogen residue,
R³-R⁵ independently of each other mean a hydrogen, halogen, cyano, or
an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be substituted,
an unsubstituted C₃-C₁₀ cycloalkyl ring, which can optionally be substituted,
an unsubstituted C₂-C₆ alkenyl or C₂-C₆ alkynyl group, which can optionally be substituted,
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally also be halogenated or else substituted with —OH,
—CN or —CO₂H,
—OR⁹, —OC(O)R⁹, —S(O)ₙR⁹ wherein n=0, 1 or 2,
—SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl residue,
an unsubstituted 5-6-membered aryl or heteroaryl ring, which can optionally be singly or multiply substituted, wherein the substituents can be selected from the group
halogen, cyano, —R⁹—OR⁹, —OC(O)R⁹, —S(O)ₙR⁹ wherein
n=0, 1 or 2, —SO₂NHR⁹, NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which can be unsubstituted or optionally up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

14. The compounds as claimed in claim 1, wherein
X means a nitrogen residue,
Y and Z mean a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
wherein the substituents can be selected from the group
halogen,
—C₁-C₄ alkyl, which can be unsubstituted or optionally substituted, —OR⁶, —OC(O)R⁶, —S(O)ₙR⁶, wherein n=0, 1 or 2,
—SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be up to quintuply halogenated or else substituted with, —CN or —CO₂H,
an unsubstituted C₃-C₁₀ cycloalkyl ring,
an unsubstituted C₂-C₆ alkenyl group, which can optionally be up to triply halogenated or else substituted with, —CN or —CO₂H,
an unsubstituted C₂-C₆ alkynyl group, which can optionally be singly halogenated or else substituted with —CN or —CO₂H and,
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally be up to quintuply halogenated or else substituted with —OH, —CN or —CO₂H, —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl group,
an unsubstituted 5-6-membered aryl or heteroaryl ring, which can optionally be singly or multiply substituted, wherein the substituents can be selected from the group
halogen,
cyano,
R⁹—OR⁹, —OC(O)R⁹, —S(O)ₙR⁹, wherein n=0, 1 or 2,
—SO₂NHR⁹, —NR⁹R¹⁰, —NHC(O)R⁹, —CO₂—R⁹ or
—C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

15. The compounds as claimed in claim 1, wherein
Y and Z mean a nitrogen residue,
X means a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
wherein the substituents can be selected from the group halogen, —C₁-C₄ alkyl, which can be unsubstituted or optionally substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be up to quintuply halogenated or else substituted with —CN or —CO₂H,
an unsubstituted C₃-C₁₀ cycloalkyl ring,
an unsubstituted C₂-C₆ alkenyl group, which can optionally be up to triply halogenated or else substituted with —CN or —CO₂H,
an unsubstituted C₂-C₆ alkynyl group, which can optionally be singly halogenated or else substituted with —CN or —CO₂H and,
an unsubstituted 5-6-membered aryl or heteroaryl ring, which is optionally singly or multiply substituted,
wherein the substituents can be selected from the group
halogen,
C₁-C₄ alkyl, which is unsubstituted and can optionally be up to quintuply halogenated or else substituted with —OH, —CN or —CO₂H, —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
an unsubstituted C₁-C₆ alkyl group, which can optionally be up to quintuply halogenated,
an unsubstituted C₃-C₈ cycloalkyl group,
an unsubstituted 5-6-membered aryl or heteroaryl ring, which can optionally be singly or multiply substituted, wherein the substituents can be selected from the group
halogen,
cyano,
R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

16. The compounds as claimed in claim 1, wherein
X and Z mean a nitrogen residue,
Y means a carbon residue —C—R⁸,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
wherein the substituents can be selected from the group halogen, —C₁-C₄ alkyl, which can be unsubstituted or optionally substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
an OR⁶, OC(O)R⁶, S(O)NR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷ group,
an unsubstituted C₁-C₆ alkyl group, which can optionally be up to quintuply halogenated or else substituted with —CN or —CO₂H,
an unsubstituted C₃-C₁₀ cycloalkyl ring,
an unsubstituted C₂-C₆ alkenyl group, which can optionally be up to triply halogenated or else substituted with —CN or —CO₂H,
an unsubstituted C₂-C₆ alkynyl group, which can optionally be singly halogenated or else substituted with —CN or —CO₂H, and
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally be up to quintuply halogenated or else substituted with —OH, —CN or —CO₂H, —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ means a hydrogen,
a C₁-C₆ alkyl group, which can be unsubstituted or optionally up to quintuply halogenated,
a C₃-C₈ cycloalkyl group,
a 5-6-membered aryl or heteroaryl ring, which can optionally be singly or multiply substituted, wherein the substituents can be selected from the group
halogen,
cyano,
an unsubstituted C₃-C₈ cycloalkyl group,
R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

17. The compounds as claimed in claim 1, wherein
X and Y mean a carbon residue —C—R⁸,
Z means a nitrogen residue,
R⁸ means a hydrogen,
R¹ means a phenyl, thiophen-2-yl, thiophen-3-yl, furan-2-yl, furan-3-yl, pyrid-3-yl or pyrid-4-yl ring, which is optionally singly to triply substituted,
wherein the substituents can be selected from the group halogen, —C₁-C₄ alkyl, which can be unsubstituted or optionally substituted, —OR⁶, —OC(O)R⁶, —S(O)ₙR⁶, wherein n=0, 1 or 2, —SO₂NHR⁶, —SO₂NHC(O)R⁶, NR⁶R⁷, —NHC(O)R⁶, —CN, —CO₂—R⁶, —C(O)—N—R⁶R⁷, —C(O)R⁶ or —C(OH)R⁶R⁷,
R² means a hydrogen residue,
R³-R⁵ independently of each other mean a hydrogen, halogen, cyano, or
an OR⁶, OC(O)R⁶, S(O)ₙR⁶, wherein n=0, 1 or 2, SO₂NHR⁶, SO₂NHC(O)R⁶, NR⁶R⁷, NHC(O)R⁶, CH₂NR⁶R⁷, CH₂NHC(O)R⁶, C(OH)R⁶R⁷, C(O)R⁶, CO₂R⁶ or C(O)NR⁶R⁷— group,
an unsubstituted C₁-C₆ alkyl group, which can optionally be up to quintuply halogenated or else substituted with —CN or —CO₂H,
an unsubstituted C₃-C₁₀ cycloalkyl ring,
an unsubstituted C₂-C₆ alkenyl group, which can optionally be up to triply halogenated or else substituted with —CN or —CO₂H,
an unsubstituted C₂-C₆ alkynyl group, which can optionally be singly halogenated or else substituted with —CN or —CO₂H and,
an unsubstituted 5-6-membered aryl or heteroaryl ring, which is optionally singly or multiply substituted,
wherein the substituents can be selected from the group
halogen,
—C₁-C₄ alkyl, which is unsubstituted and can optionally be up to quintuply halogenated or else substituted with —OH, —CN or —CO₂H, —OR⁹, —OC(O)R⁹, —S(O)ₙR⁹, wherein n=0, 1 or 2, —SO₂NHR⁹, —SO₂NHC(O)R⁹, NR⁹R¹⁰, —NHC(O)R⁹, —CN, —CO₂—R⁹, —C(O)—N—R⁹R¹⁰, —C(O)R⁹ or —C(OH)R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group,
R⁶, R⁷ mean a hydrogen,
a C₁-C₆ alkyl group, which is unsubstituted or can optionally be up to quintuply halogenated,
a 5-6-membered aryl or heteroaryl ring, which can optionally be singly or multiply substituted, wherein the substituents can be selected from the group
halogen,
cyano,
a C₃-C₈ cycloalkyl group,
R⁹, —OR⁹, —OC(O)R⁹, —S(O)ₙR⁹, wherein n=0, 1 or 2, —SO₂NHR⁹, —NR⁹R¹⁰, —NHC(O)R⁹, —CO₂—R⁹ or —C(O)—N—R⁹R¹⁰,
wherein the 5-6-membered aryl or heteroaryl ring can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl group, or
R⁶, R⁷ together form a 3-8-membered ring,
R⁹, R¹⁰ independently of each other mean hydrogen, a C₁-C₄ alkyl group, which is unsubstituted or can optionally be up to quintuply fluorinated,
an unsubstituted C₂-C₄ alkenyl group, which can optionally be up to triply fluorinated,
an unsubstituted C₂-C₄ alkynyl group, which can optionally be singly fluorinated,
an unsubstituted C₃-C₆ cycloalkyl group,
an unsubstituted 5-6-membered aryl or heteroaryl ring, which can for example, but not exclusively, be a phenyl, pyridinyl, pyrimidinyl, furanyl, thiophenyl, oxazolyl, isoxazolyl, thiazolyl, pyrrolyl, pyrazolyl, imidazolyl, pyrazinyl, pyridazinyl, triazolyl or tetrazolyl ring, which can optionally be up to doubly substituted with fluorine, chlorine or trifluoromethyl, or
R⁹, R¹⁰ together form a 3-8-membered ring.

18. The compounds as claimed in claim 1, selected from a group which contains the following compounds:
1-[1-(7-chloro-quinolin-4-yl)-piperidin-4-yl]-3-(4-methylsulfanyl-phenyl)-thiourea
1-[1-(7-chloro-quinolin-4-yl)-piperidin-4-yl]-3-phenyl-thiourea
1-(3-chloro-phenyl)-3-[1-(7-chloro-quinolin-4-yl)-piperidin-4-yl]-thiourea
1-(2-chloro-phenyl)-3-[1-(7-chloro-quinolin-4-yl)-piperidin-4-yl]-thiourea
1-(1-(7-chloro-quinolin-4-yl)-piperidin-4-yl)-3-(4-methoxy-phenyl)-thiourea
1-(1-(7-chloro-quinolin-4-yl)-piperidin-4-yl)-3-p-tolyl-thiourea
1-(1-(7-chloro-quinolin-4-yl)-piperidin-4-yl)-3-o-tolyl-thiourea
1-(2-bromo-phenyl)-3-(1-(7-chloro-quinolin-4-yl)-piperidin-4-yl)-thiourea
1-(3-bromo-phenyl)-3-(1-(7-chloro-quinolin-4-yl)-piperidin-4-yl)-thiourea
1-(1-(7-chloro-quinolin-4-yl)-piperidin-4-yl)-3-(3-methoxy-phenyl)-thiourea methyl 4-(3-(1-(7-chloro-quinolin-4-yl)-piperidin-4-yl)-thioureido)-benzoate
1-(1-(7-chloro-quinolin-4-yl)-piperidin-4-yl)-3-(3-cyano-phenyl)-thiourea
1-(1-(7-chloro-quinolin-4-yl)-piperidin-4-yl)-3-(4-cyano-phenyl)-thiourea
1-(1-(7-chloro-quinolin-4-yl)-piperidin-4-yl)-3-(2-methylsulfanyl-phenyl)-thiourea
1-(1-chloro-quinolin-4-yl)-piperidin-4-yl)-3-(2,3-dichloro-phenyl)-thiourea
1-(1-(7-chloro-quinolin-4-yl)-piperidin-4-yl)-3-(3-methylsulfanyl-phenyl)-thiourea
methyl 3-(3-(1-(7-chloro-quinolin-4-yl)-piperidin-4-yl)-thioureido)-benzoate
1-(1-(7-chloro-quinolin-4-yl)-piperidin-4-yl)-3-(2-trifluoromethoxy-phenyl)-thiourea
1-[1-(8-chloro-quinolin-4-yl)-piperidin-4-yl]-3-(4-methylsulfanyl-phenyl)-thiourea
1-[1-(8-chloro-quinolin-4-yl)-piperidin-4-yl]-3-phenyl-thiourea
1-(3-chloro-phenyl)-3-[1-(8-chloro-quinolin-4-yl)-piperidin-4-yl]-thiourea
1-(2-chloro-phenyl)-3-[1-(8-chloro-quinolin-4-yl)-piperidin-4-yl]-thiourea
1-(1-(8-chloro-quinolin-4-yl)-piperidin-4-yl)-3-(4-methoxy-phenyl)-thiourea
1-(1-(8-chloro-quinolin-4-yl)-piperidin-4-yl)-3-p-tolyl-thiourea
1-(1-(8-chloro-quinolin-4-yl)-piperidin-4-yl)-3-o-tolyl-thiourea
1-(2-bromo-phenyl)-3-(1-(8-chloro-quinolin-4-yl)-piperidin-4-yl)-thiourea
1-(3-bromo-phenyl)-3-(1-(8-chloro-quinolin-4-yl)-piperidin-4-yl)-thiourea
1-(8-chloro-quinolin-4-yl)-piperidin-4-yl)-3-(3-methoxy-phenyl)-thiourea
methyl 4-(3-(1-(8-chloro-quinolin-4-yl)-piperidin-4-yl)-thioureido)-benzoate
1-(1-(8-chloro-quinolin-4-yl)-piperidin-4-yl)-3-(3-cyano-phenyl)-thiourea
1-(8-chloro-quinolin-4-yl)-piperidin-4-yl)-3-(4-cyano-phenyl)-thiourea
1-(1-(8-chloro-quinolin-4-yl)-piperidin-4-yl)-3-(2-methylsulfanyl-phenyl)-thiourea
1-(1-(8-chloro-quinolin-4-yl)-piperidin-4-yl)-3-(2,3-dichloro-phenyl)-thiourea
1-(1-(8-chloro-quinolin-4-yl)-piperidin-4-yl)-3-(3-methylsulfanyl-phenyl)-thiourea
methyl 3-(3-(1-(8-chloro-quinolin-4-yl)-piperidin-4-yl)-thioureido)-benzoate
1-(1-(8-chloro-quinolin-4-yl)-piperidin-4-yl)-3-(2-trifluoromethoxy-phenyl)-thiourea
1-[1-(6-chloro-quinolin-4-yl)-piperidin-4-yl]-3-(4-methylsulfanyl-phenyl)-thiourea
1-[1-(6-chloro-quinolin-4-yl)-piperidin-4-yl]-3-phenyl-thiourea
1-(3-chloro-phenyl)-3-[1-(6-chloro-quinolin-4-yl)-piperidin-4-yl]-thiourea
1-(2-chloro-phenyl)-3-[1-(6-chloro-quinolin-4-yl)-piperidin-4-yl]-thiourea
1-(1-(6-chloro-quinolin-4-yl)-piperidin-4-yl)-3-(4-methoxy-phenyl)-thiourea
1-(1-(6-chloro-quinolin-4-yl)-piperidin-4-yl)-3-p-tolyl-thiourea
1-(1-(6-chloro-quinolin-4-yl)-piperidin-4-yl)-3-o-tolyl-thiourea
1-(2-bromo-phenyl)-3-(1-(6-chloro-quinolin-4-yl)-piperidin-4-yl)-thiourea
1-(3-bromo-phenyl)-3-(1-(6-chloro-quinolin-4-yl)-piperidin-4-yl)-thiourea
1-(1-(6-chloro-quinolin-4-yl)-piperidin-4-yl)-3-(3-methoxy-phenyl)-thiourea
methyl 4-(3-(1-(6-chloro-quinolin-4-yl)-piperidin-4-yl)-thioureido)-benzoate
1-(6-chloro-quinolin-4-yl)-piperidin-4-yl)-3-(3-cyano-phenyl)-thiourea
1-(1-(6-chloro-quinolin-4-yl)-piperidin-4-yl)-3-(4-cyano-phenyl)-thiourea
1-(1-(6-chloro-quinolin-4-yl)-piperidin-4-yl)-3-(2-methylsulfanyl-phenyl)-thiourea
1-(1-(6-chloro-quinolin-4-yl)-piperidin-4-yl)-3-(2,3-dichloro-phenyl)-thiourea
(1-(6-chloro-quinolin-4-yl)-piperidin-4-yl)-3-(3-methylsulfanyl-phenyl)-thiourea
methyl 3-(3-(1-(6-chloro-quinolin-4-yl)-piperidin-4-yl)-thioureido)-benzoate
1-(1-(6-chloro-quinolin-4-yl)-piperidin-4-yl)-3-(2-trifluoromethoxy-phenyl)-thiourea
1-[1-(6-bromo-phthalazin-1-yl)-piperidin-4-yl]-3-(4-methylsulfanyl-phenyl)-thiourea
1-[1-(6-bromo-phthalazin-1-yl)-piperidin-4-yl]-3-phenyl-thiourea
1-(3-chloro-phenyl)-3-[1-(6-bromo-phthalazin-1-yl)-piperidin-4-yl]-thiourea
1-(2-chloro-phenyl)-3-[1-(6-bromo-phthalazin-1-yl)-piperidin-4-yl]-thiourea
1-(1-(6-bromo-phthalazin-1-yl)-piperidin-4-yl)-3-(4-methoxy-phenyl)-thiourea
1-(1-(6-bromo-phthalazin-1-yl)-piperidin-4-yl)-3-p-tolyl-thiourea
1-(1-(6-bromo-phthalazin-1-yl)-piperidin-4-yl)-3-o-tolyl-thiourea
1-(2-bromo-phenyl)-3-(1-(6-bromo-phthalazin-1-yl)-piperidin-4-yl)-thiourea
1-(3-bromo-phenyl)-3-(1-(6-bromo-phthalazin-1-yl)-piperidin-4-yl)-thiourea
1-(1-(6-bromo-phthalazin-1-yl)-piperidin-4-yl)-3-(3-methoxy-phenyl)-thiourea
methyl 4-(3-(1-(6-bromo-phthalazin-1-yl)-piperidin-4-yl)-thioureido)-benzoate
1-(1-(6-bromo-phthalazin-1-yl)-piperidin-4-yl)-3-(3-cyano-phenyl)-thiourea
1-(1-(6-bromo-phthalazin-1-yl)-piperidin-4-yl)-3-(4-cyano-phenyl)-thiourea
1-(1-(6-bromo-phthalazin-1-yl)-piperidin-4-yl)-3-(2-methylsulfanyl-phenyl)-thiourea
1-(1-(6-bromo-phthalazin-1-yl)-piperidin-4-yl)-3-(2,3-dichloro-phenyl)-thiourea
1-(1-(6-bromo-phthalazin-1-yl)-piperidin-4-yl)-3-(3-methylsulfanyl-phenyl)-thiourea
methyl 3-(3-(1-(6-bromo-phthalazin-1-yl)-piperidin-4-yl)-thioureido)-benzoate
1-(1-(6-bromo-phthalazin-1-yl)-piperidin-4-yl)-3-(2-trifluoromethoxy-phenyl)-thiourea
1-[1-(7-bromo-phthalazin-1-yl)-piperidin-4-yl]-3-(4-methylsulfanyl-phenyl)-thiourea
1-[1-(7-bromo-phthalazin-1-yl)-piperidin-4-yl]-3-(4-methylsulfanyl-phenyl)-thiourea
1-[1-(7-bromo-phthalazin-1-yl)-piperidin-4-yl]-3-phenyl-thiourea
1-(3-chloro-phenyl)-3-[1-(7-bromo-phthalazin-1-yl)-piperidin-4-yl]-thiourea 1-(2-chloro-phenyl)-3-[1-(7-bromo-phthalazin-1-yl)-piperidin-4-yl]-thiourea
1-(1-(7-bromo-phthalazin-1-yl)-piperidin-4-yl)-3-(4-methoxy-phenyl)-thiourea
1-(1-(7-bromo-phthalazin-1-yl)-piperidin-4-yl)-3-p-tolyl-thiourea
1-(1-(7-bromo-phthalazin-1-yl)-piperidin-4-yl)-3-o-tolyl-thiourea
1-(2-bromo-phenyl)-3-(1-(7-bromo-phthalazin-1-yl)-piperidin-4-yl)-thiourea
1-(3-bromo-phenyl)-3-(1-(7-bromo-phthalazin-1-yl)-piperidin-4-yl)-thiourea
1-(1-(7-bromo-phthalazin-1-yl)-piperidin-4-yl)-3-(3-methoxy-phenyl)-thiourea
methyl 4-(3-(1-(7-bromo-phthalazin-1-yl)-piperidin-4-yl)-thioureido)-benzoate
1-(1-(7-bromo-phthalazin-1-yl)-piperidin-4-yl)-3-(3-cyano-phenyl)-thiourea
1-(1-(7-bromo-phthalazin-1-yl)-piperidin-4-yl)-3-(4-cyano-phenyl)-thiourea
1-(1-(7-bromo-phthalazin-1-yl)-piperidin-4-yl)-3-(2-methylsulfanyl-phenyl)-thiourea
1-(1-(7-bromo-phthalazin-1-yl)-piperidin-4-yl)-3-(2,3-dichloro-phenyl)-thiourea
1-(1-(7-bromo-phthalazin-1-yl)-piperidin-4-yl)-3-(3-methylsulfanyl-phenyl)-thiourea
methyl 3-(3-(1-(7-bromo-phthalazin-1-yl)-piperidin-4-yl)-thioureido)-benzoate
1-(1-(7-bromo-phthalazin-1-yl)-piperidin-4-yl)-3-(2-trifluoromethoxy-phenyl)-thiourea
1-[1-(7-chloro-quinazol in-4-yl)-piperidin-4-yl]-3-(4-methylsulfanyl-phenyl)-thiourea
1-[1-(7-chloro-quinazolin-4-yl)-piperidin-4-yl]-3-phenyl-thiourea
1-(3-chloro-phenyl)-3-[1-(7-chloro-quinazol in-4-yl)-piperidin-4-yl]-thiourea
1-(2-chloro-phenyl)-3-[1-(7-chloro-quinazol in-4-yl)-piperidin-4-yl]-thiourea
1-(1-(7-chloro-quinazolin-4-yl)-piperidin-4-yl)-3-(4-methoxy-phenyl)-thiourea
1-(1-(7-chloro-quinazolin-4-yl)-piperidin-4-yl)-3-p-tolyl-thiourea
1-(1-(7-chloro-quinazol in-4-yl)-piperidin-4-yl)-3-o-tolyl-thiourea
1-(2-bromo-phenyl)-3-(1-(7-chloro-quinazolin-4-yl)-piperidin-4-yl)-thiourea
1-(3-bromo-phenyl)-3-(1-(7-chloro-quinazol in-4-yl)-piperidin-4-yl)-thiourea
1-(1-(7-chloro-quinazol in-4-yl)-piperidin-4-yl)-3-(3-methoxy-phenyl)-thiourea
methyl 4-(3-(1-(7-chloro-quinazol in-4-yl)-piperidin-4-yl)-thioureido)-benzoate
1-(1-(7-chloro-quinazolin-4-yl)-piperidin-4-yl)-3-(3-cyano-phenyl)-thiourea
1-(1-(7-chloro-quinazol in-4-yl)-piperidin-4-yl)-3-(4-cyano-phenyl)-thiourea
1-(1-(7-chloro-quinazol in-4-yl)-piperidin-4-yl)-3-(2-methylsulfanyl-phenyl)-thiourea
1-(1-(7-chloro-quinazol in-4-yl)-piperidin-4-yl)-3-(2,3-dichloro-phenyl)-thiourea
1-(1-(7-chloro-quinazol in-4-yl)-piperidin-4-yl)-3-(3-methylsulfanyl-phenyl)-thiourea
methyl 3-(3-(1-(7-chloro-quinazolin-4-yl)-piperidin-4-yl)-thioureido)-benzoate
1-(1-(7-chloro-quinazol in-4-yl)-piperidin-4-yl)-3-(2-trifluoromethoxy-phenyl)-thiourea
1-(1-isoquinolin-1-yl-piperidin-4-yl)-3-(4-methylsulfanyl-phenyl)-thiourea
1-(1-isoquinolin-1-yl-piperidin-4-yl)-3-phenyl-thiourea
1-(3-chloro-phenyl)-3-(1-isoquinolin-1-yl-piperidin-4-yl)-thiourea
1-(2-chloro-phenyl)-3-(1-isoquinolin-1-yl-piperidin-4-yl)-thiourea
1-(1-isoquinolin-1-yl-piperidin-4-yl)-3-(4-methoxy-phenyl)-thiourea
1-(1-isoquinolin-1-yl-piperidin-4-yl)-3-p-tolyl-thiourea
1-(1-isoquinolin-1-yl-piperidin-4-yl)-3-o-tolyl-thiourea
1-(2-bromo-phenyl)-3-(1-isoquinolin-1-yl-piperidin-4-yl)-thiourea
1-(3-bromo-phenyl)-3-(1-isoquinolin-1-yl-piperidin-4-yl)-thiourea
1-(1-isoquinolin-1-yl-piperidin-4-yl)-3-(3-methoxy-phenyl)-thiourea
methyl 4-(3-(1-isoquinolin-1-yl-piperidin-4-yl)-thioureido)-benzoate
1-(1-isoquinolin-1-yl-piperidin-4-yl)-3-(3-cyano-phenyl)-thiourea
1-(1-isoquinolin-1-yl-piperidin-4-yl)-3-(4-cyano-phenyl)-thiourea
1-(1-isoquinolin-1-yl-piperidin-4-yl)-3-(2-methylsulfanyl-phenyl)-thiourea
1-(1-isoquinolin-1-yl-piperidin-4-yl)-3-(2,3-dichloro-phenyl)-thiourea
1-(1-isoquinolin-1-yl-piperidin-4-yl)-3-(3-methylsulfanyl-phenyl)-thiourea
methyl 3-(3-(1-isoquinolin-1-yl-piperidin-4-yl)-thioureido)-benzoate
1-(1-isoquinolin-1-yl-piperidin-4-yl)-3-(2-trifluoromethoxy-phenyl)-thiourea

19. A use of the compounds as claimed in claim 1 for the production of drugs which contain at least one of the compounds of the formula 1.

20. A drug as claimed in claim 19 with suitable formulation and carrier substances.

21. The use of the drug as claimed in claim 1, characterized in that the drug is used for the treatment and prophylaxis of diseases.

22. The use as claimed in claim 21, for the treatment and prophylaxis of diseases which are connected with the EP₂ receptor.

23. The use as claimed in claim 21 for the treatment and prophylaxis of fertility disorders.

24. The use as claimed in claim 21 for the treatment and prophylaxis of menstrual problems.

25. The use as claimed in claim 21 for the treatment and prophylaxis of endometriosis.

26. The use of the compounds as claimed in claim 1 for the modulation of the EP₂ receptor.

27. The use as claimed in claim 21 for the treatment and prophylaxis of pain.

28. The use of the compounds as claimed in claim 1 and of the for fertility control.

29. The use as claimed in claim 21 for the treatment and prophylaxis of osteoporosis.

30. The use as claimed in claim 21 for the treatment and prophylaxis of cancer.

31. The use of the compounds of the general formula I, as claimed in claim 1, in the form of a pharmaceutical preparation for enteral, parenteral, vaginal and oral administration.

32. A method for the treatment and prophylaxis of diseases which are connected with the EP₂ receptor comprising administering a compound of claim 1.
